SEQUENCE LISTING (8 sequences)

SEQ ID NO: 1 - protein, 142 aa, Artificial Sequence
  Hemoglobin subunit alpha
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 2 - DNA, 575 bp, Artificial Sequence
  Hemoglobin subunit alpha
actcttctggtccccacagactcagagagaacccaccatggtgctgtctcctgccgacaagaccaacgtcaaggccgcctggggcaaggttggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagcagttcctcctgccagatgggcctcccaacgggccctcctcccctccttgcaccggcccttcctggtctttgaataaagtctgagtgggcggc

SEQ ID NO: 3 - protein, 147 aa, Artificial Sequence
  Hemoglobin subunit beta
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 4 - DNA, 628 bp, Artificial Sequence
  Hemoglobin subunit beta
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 5 - protein, 147 aa, Artificial Sequence
  Hemoglobin subunit gamma
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTGVASALSSRYH

SEQ ID NO: 6 - DNA, 586 bp, Artificial Sequence
  Hemoglobin subunit gamma
acactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacaca

SEQ ID NO: 7 - protein, 147 aa, Artificial Sequence
  Hemoglobin subunit gamma
MGHFTEEDKATITSLWGKVNVEDAGGETLGRLLVVYPWTQRFFDSFGNLSSASAIMGNPKVKAHGKKVLTSLGDATKHLDDLKGTFAQLSELHCDKLHVDPENFKLLGNVLVTVLAIHFGKEFTPEVQASWQKMVTAVASALSSRYH

SEQ ID NO: 8 - DNA, 584 bp, Artificial Sequence
  Hemoglobin subunit gamma
acactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcac

(19) United States
(12) Patent Application Publication
(10) Pub. No.: US 2024/0294609 A1
(43) Pub. Date: Sep. 5, 2024

(54) STABILIZED HEMOGLOBIN COMPOSITIONS AND PHARMACEUTICAL FORMULATIONS THEREOF

(71) Applicant: Medical Technology Associates II, Inc., Malvern, PA (US)

(72) Inventors: Carl W. RAUSCH, Malvern, PA (US); Hubert HO, Malvern, PA (US); Tiffany RAU, Malvern, PA (US)

(21) Appl. No.: 17/768,147

(22) PCT Filed: Oct. 9, 2020
(86) PCT No.: PCT/US2020/054985

§ 371 Date: Apr. 11, 2022

Related U.S. Application Data
(60) Provisional application No. 62/914,118, filed on Oct. 11, 2019.

(51) Int. Cl. C07K 14/805; A61K 9/00; A61K 38/00; A61K 47/02; A61K 47/18; A61P 7/06

(52) U.S. Cl. CPC C07K 14/805; A61K 9/0026; A61K 47/02; A61K 47/183; A61P 7/06; A61K 38/00

(57) ABSTRACT

The present disclosure provides stabilized hemoglobin compositions, uses thereof, and devices for administration thereof. The stabilized hemoglobin compositions may be useful in the treatment of various anemic or traumatic conditions involving inadequate blood or oxygen supply.

### CROSS-REFERENCE TO RELATED APPLICATIONS

This application claims the benefit of U.S. Provisional Application No. 62/914,118, filed on Oct. 11, 2019.

### INCORPORATION OF THE SEQUENCE LISTING

The contents of the text file submitted electronically herewith are incorporated herein by reference in their entirety: a computer readable format copy of the Sequence Listing (filename: MTAI_001_00 US_SeqList_ST25.TXT, date recorded: Oct. 11, 2019, file size: 9,370 bytes).

### FIELD OF THE INVENTION

The present disclosure relates to compositions comprising hemoglobin, uses thereof, and devices for intravenous administration thereof. The compositions may be useful in treating anemia and other blood deficiency disorders.

### BACKGROUND

In human beings and mammals, hemoglobin is the iron-containing oxygen-transport metalloprotein in red blood cells that carries oxygen from the lungs to the rest of the body (i.e., the tissues). There it releases oxygen to permit aerobic respiration to provide energy to power the functions of the organism in the process of metabolism. A healthy individual has 12 to 20 grams of hemoglobin in every 100 ml of blood. Hemoglobin has an oxygen-binding capacity of 1.34 mL O₂ per gram, which increases the total blood oxygen capacity seventy-fold compared to dissolved oxygen in blood. The mammalian hemoglobin molecule can bind up to four oxygen molecules. In most vertebrates, the hemoglobin molecule is an assembly of four globular protein subunits. Each subunit is composed of a protein chain tightly associated with a non-protein prosthetic heme group. Each protein chain arranges into a set of alpha-helix structural segments connected together in a globin fold arrangement. This folding pattern contains a pocket that strongly binds the heme group.

In the treatment of trauma patients, transfusion with whole allogeneic blood is ubiquitous. However, on the worldwide scale, there is a shortage of safe and viable allogeneic donor blood, a problem that is only projected to increase over time. In addition, whole blood transfusion comes with risks, including blood-borne diseases, fatal ABO-incompatibility, systemic inflammatory response, and multiple organ failure. In addition, whole human blood has a limited shelf life of 42 days and the available quantities are insufficient for emergency situations involving numerous traumatic injuries, such as in warfare or after a natural disaster.

Numerous efforts have been made to develop hemoglobin-based oxygen carriers (HBOCs), but most hemoglobin-based blood substitutes fail clinical trials because of safety concerns, including severe vasoconstriction and increased mortality. In spite of these failures, hemoglobin-based blood substitutes remain an attractive alternative because of the biological similarity of such products to natural oxygen carriers and because of their successful use in veterinary applications.

There is an unmet need for safe hemoglobin-based blood substitutes for human treatment.

### SUMMARY

Disclosed herein is a hemoglobin based composition comprising a stabilized hemoglobin. In one aspect, the present disclosure provides a composition comprising a stabilized hemoglobin at a concentration of between 150 grams (g) per Liter (L) (g/L) and 200 g/L, inclusive of the endpoints, wherein the composition comprises less than 0.02 milligrams (mg) per milliliter (mL) (mg/mL) of dissolved oxygen. In another aspect, the present disclosure provides a composition comprising a stabilized hemoglobin, wherein the stabilized hemoglobin comprises: 20-35% of the total hemoglobin being in tetrameric form; 15-20% of the total hemoglobin being in octameric form; 40-55% of the total hemoglobin being in greater-than-octameric form; less than 5% of the total hemoglobin being in dimer form; or any combination thereof. In some embodiments, the stabilized hemoglobin is in a stabilized hemoglobin solution. In some embodiments, the stabilized hemoglobin comprises: 20-35% of the total hemoglobin being in tetrameric form, for example, 20-25%, 20-30%, 25-30%, 25-35%, or 30-35% of the total hemoglobin being in tetrameric form. In some embodiments, the stabilized hemoglobin comprises: 15-20% of the total hemoglobin being in octameric form. In some embodiments, the stabilized hemoglobin comprises: 40-55% of the total hemoglobin being in greater-than-octameric form, for example, 40-45%, 40-50%, 45-50%, 45-55%, or 50-55% of the total hemoglobin being in greater-than-octameric form. In some embodiments, the stabilized hemoglobin comprises: less than 5% of the total hemoglobin being in dimer form.

In some embodiments, the composition comprises less than 0.02 mg/mL of dissolved oxygen. In some embodiments, the stabilized hemoglobin is at a concentration of between 70 and 200 grams per Liter (g/L) inclusive of the endpoints. In some embodiments, the stabilized hemoglobin is at a concentration of between 150 and 200 g/L inclusive of the endpoints. In some embodiments, the stabilized hemoglobin is at a concentration of 70 g/L to 200 g/L. In some embodiments, the stabilized hemoglobin is at a concentration of at least 70 g/L. In some embodiments, the stabilized hemoglobin is at a concentration of at most 200 g/L. In some embodiments, the stabilized hemoglobin is at a concentration of 70 g/L to 80 g/L, 70 g/L to 90 g/L, 70 g/L to 100 g/L, 70 g/L to 120 g/L, 70 g/L to 140 g/L, 70 g/L to 160 g/L, 70 g/L to 180 g/L, 70 g/L to 200 g/L, 80 g/L to 90 g/L, 80 g/L to 100 g/L, 80 g/L to 120 g/L, 80 g/L to 140 g/L, 80 g/L to 160 g/L, 80 g/L to 180 g/L, 80 g/L to 200 g/L, 90 g/L to 100 g/L, 90 g/L to 120 g/L, 90 g/L to 140 g/L, 90 g/L to 160 g/L, 90 g/L to 180 g/L, 90 g/L to 200 g/L, 100 g/L to 120 g/L, 100 g/L to 140 g/L, 100 g/L to 160 g/L, 100 g/L to 180 g/L, 100 g/L to 200 g/L, 120 g/L to 140 g/L, 120 g/L to 160 g/L, 120 g/L to 180 g/L, 120 g/L to 200 g/L, 140 g/L to 160 g/L, 140 g/L to 180 g/L, 140 g/L to 200 g/L, 160 g/L to 180 g/L, 160 g/L to 200 g/L, or 180 g/L to 200 g/L. In some embodiments, the stabilized hemoglobin is at a concentration of 70 g/L, 80 g/L, 90 g/L, 100 g/L, 120 g/L, 140 g/L, 160 g/L, 180 g/L, or 200 g/L.

In some embodiments, the stabilized hemoglobin is stabilized by contacting at least one stabilizing agent selected from a group consisting of: glutaraldehyde, succindialdehyde, activated forms of polyoxyethylene and dextran, α-hydroxy aldehydes, glycolaldehyde, N-maleimido-6-aminocaproyl-(2′-nitro, 4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl) cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl) butyrate, sulfosuccinimidyl 4-(p-maleimidophenyl)butyrate, 1-ethyl-3-(3-dimethylaminopropyl)carbodiimide hydrochloride, N,N′-phenylene dimaleimide, a bis-imidate compound, an acyl diazide compound, an aryl dihalide compound, and combinations thereof.

In some embodiments, the composition further comprises a formulation buffer comprising one or more of borate, anti-oxidants, and electrolytes. In some embodiments, the borate is reduced. In some embodiments, the anti-oxidants comprise N-acetyl-L-cysteine. In some embodiments, the electrolytes comprise Na, Cl, and/or K. In some embodiments, the composition comprises fewer than 0.05 endotoxin units (EU) per milliliter (mL) (EU/mL). In some embodiments, the hemoglobin comprises hemoglobin isolated or derived from a human, a human cell or a human cell line. In some embodiments, the hemoglobin is isolated or derived from no more than 100 variable sources. In some embodiments, the hemoglobin is isolated or derived from less than 90, 80, 70, 60, 50, 40, 30, 20, 10, 5, or 3 variable sources. In some embodiments, the hemoglobin is isolated or derived from a single source. In some embodiments, the hemoglobin is isolated or derived from harvested red blood cells. In some embodiments, the hemoglobin is isolated or derived from harvested red blood cells within 15, 10, 5, or 2 days after harvest. In some embodiments, the hemoglobin comprises hemoglobin isolated or derived from a non-human animal, a non-human cell or a non-human cell line. In some embodiments, the hemoglobin is isolated or derived from harvested red blood cells within 15, 10, 5, or 2 days after harvest. In some embodiments, the non-human animal is a nonhuman vertebrate, a non-human primate, a cetacean, a mammal, a reptile, a bird, an amphibian, or a fish. In some embodiments, the non-human animal is a bovine species. In some embodiments, the non-human animal is an ovine species. In some embodiments, the non-human animal is a mustelid, a captive mustelid, a rodent, a captive rodent, a raptor, or a captive bird. In some embodiments, the captive bird is of the order Psittaciformes, Passeriformes or Columbiformes. In some embodiments, the non-human animal is not a squab that is raised for food.

In some embodiments, the hemoglobin comprises: (a) a subunit alpha (α), wherein the subunit α comprises the amino acid sequence of:

```
(SEQ ID NO: 1)
    1 MVLSPADKTN VKAAWGKVGA HAGEYGAEAL
        ERMFLSFPTT KTYFPHFDLS HGSAQVKGHG
  61 KKVADALTNA VAHVDDMPNA LSALSDLHAH
        KLRVDPVNFK LLSHCLLVTL AAHLPAEFTP
121 AVHASLDKFL ASVSTVLTSK YR,
```

or a sequence having at least 90% identity to the sequence of SEQ ID NO: 1, or wherein the subunit α is encoded by the nucleic acid sequence of

```
(SEQ ID NO: 2)
    1 actcttctgg tccccacaga ctcagagaga
        acccaccatg gtgctgtctc ctgccgacaa
  61 gaccaacgtc aaggccgcct ggggcaaggt
        tggcgcgcac gctggcgagt atggtgcgga
121 ggccctggag aggatgttcc tgtccttccc
        caccaccaag acctacttcc cgcacttcga
181 cctgagccac ggctctgccc aggttaaggg
        ccacggcaag aaggtggccg acgcgctgac
241 caacgccgtg gcgcacgtgg acgacatgcc
        caacgcgctg tccgccctga gcgacctgca
301 cgcgcacaag cttcgggtgg acccggtcaa
        cttcaagctc ctaagccact gcctgctggt
361 gaccctggcc gcccacctcc ccgccgagtt
        cacccctgcg gtgcacgcct ccctggacaa
421 gttcctggct tctgtgagca ccgtgctgac
        ctccaaatac cgttaagctg gagcctcggt
481 agcagttcct cctgccagat gggcctccca
        acgggccctc ctcccctcct tgcaccggcc
541 cttcctggtc tttgaataaa gtctgagtgg
        gcggc,
```

or a sequence having at least 90% identity to the sequence of SEQ ID NO: 2; or (b) a subunit beta (β), wherein the subunit β comprises the amino acid sequence of

```
(SEQ ID NO: 3)
    1 MVHLTPEEKS AVTALWGKVN VDEVGGEALG
        RLLVVYPWTQ RFFESFGDLS TPDAVMGNPK
  61 VKAHGKKVLG AFSDGLAHLD NLKGTFATLS
        ELHCDKLHVD PENFRLLGNV LVCVLAHHFG
121 KEFTPPVQAA YQKVVAGVAN ALAHKYH,
```

or a sequence having at least 90% identity to the sequence of SEQ ID NO: 3,
or wherein the subunit β is encoded by the nucleic acid sequence of

```
(SEQ ID NO: 4)
    1 acatttgctt ctgacacaac tgtgttcact
        agcaacctca aacagacacc atggtgcatc
  61 tgactcctga ggagaagtct gccgttactg
        ccctgtgggg caaggtgaac gtggatgaag
121 ttggtggtga ggccctgggc aggctgctgg
        tggtctaccc ttggacccag aggttctttg
181 agtcctttgg ggatctgtcc actcctgatg
        ctgttatggg caaccctaag gtgaaggctc
241 atggcaagaa agtgctcggt gcctttagtg
        atggcctggc tcacctggac aacctcaagg
301 gcacctttgc cacactgagt gagctgcact
        gtgacaagct gcacgtggat cctgagaact
361 tcaggctcct gggcaacgtg ctggtctgtg
        tgctggccca tcactttggc aaagaattca
421 ccccaccagt gcaggctgcc tatcagaaag
        tggtggctgg tgtggctaat gccctggccc
481 acaagtatca ctaagctcgc tttcttgctg
        tccaatttct attaaaggtt cctttgttcc
541 ctaagtccaa ctactaaact gggggatatt
        atgaagggcc ttgagcatct ggattctgcc
601 taataaaaaa catttatttt cattgcaa,
```

or a sequence having at least 90% identity to the sequence of SEQ ID NO: 4;
(c) a subunit gamma (γ), wherein the subunit γ comprises the amino acid sequence of

```
(SEQ ID NO: 5)
  1 MGHFTEEDKA TITSLWGKVN VEDAGGETLG
      RLLVVYPWTQ RFFDSFGNLS SASAIMGNPK
61 VKAHGKKVLT SLGDAIKHLD DLKGTFAQLS
      ELHCDKLHVD PENFKLLGNV LVTVLAIHFG
121 KEFTPEVQAS WQKMVTGVAS ALSSRYH,
```

or a sequence having at least 90% identity to the sequence of SEQ ID NO: 5 or wherein the subunit γ is encoded by the nucleic acid sequence of

```
(SEQ ID NO: 6)
    1 acactcgctt ctggaacgtc tgaggttatc
        aataagctcc tagtccagac gccatgggtc
  61 atttcacaga ggaggacaag gctactatca
        caagcctgtg gggcaaggtg aatgtggaag
121 atgctggagg agaaaccctg ggaaggctcc
        tggttgtcta cccatggacc cagaggttct
181 ttgacagctt tggcaacctg tcctctgcct
        ctgccatcat gggcaacccc aaagtcaagg
241 cacatggcaa gaaggtgctg acttccttgg
        gagatgccat aaagcacctg gatgatctca
301 agggcacctt tgcccagctg agtgaactgc
        actgtgacaa gctgcatgtg gatcctgaga
361 acttcaagct cctgggaaat gtgctggtga
        ccgttttggc aatccatttc ggcaaagaat
421 tcacccctga ggtgcaggct tcctggcaga
        agatggtgac tggagtggcc agtgccctgt
481 cctccagata ccactgagct cactgcccat
        gatgcagagc tttcaaggat aggctttatt
541 ctgcaagcaa tcaaataata aatctattct
        gctaagagat cacaca,
```

or a sequence having at least 90% identity to the sequence of SEQ ID NO: 6; or
(d) a subunit gamma (γ), wherein the subunit γ comprises the amino acid sequence of

```
(SEQ ID NO: 7)
    1 MGHFTEEDKA TITSLWGKVN VEDAGGETLG
        RLLVVYPWTQ RFFDSFGNLS SASAIMGNPK
  61 VKAHGKKVLT SLGDATKHLD DLKGTFAQLS
        ELHCDKLHVD PENFKLLGNV LVTVLAIHFG
121 KEFTPEVQAS WQKMVTAVAS ALSSRYH,
```

or a sequence having at least 90% identity to the sequence of SEQ ID NO: 7 or wherein the subunit γ is encoded by the nucleic acid sequence of

```
(SEQ ID NO: 8)
    1 acactcgctt ctggaacgtc tgaggttatc
        aataagctcc tagtccagac gccatgggtc
  61 atttcacaga ggaggacaag gctactatca
        caagcctgtg gggcaaggtg aatgtggaag
121 atgctggagg agaaaccctg ggaaggctcc
        tggttgtcta cccatggacc cagaggttct
181 ttgacagctt tggcaacctg tcctctgcct
        ctgccatcat gggcaacccc aaagtcaagg
241 cacatggcaa gaaggtgctg acttccttgg
        gagatgccac aaagcacctg gatgatctca
301 agggcacctt tgcccagctg agtgaactgc
        actgtgacaa gctgcatgtg gatcctgaga
361 acttcaagct cctgggaaat gtgctggtga
        ccgttttggc aatccatttc ggcaaagaat
421 tcacccctga ggtgcaggct tcctggcaga
        agatggtgac tgcagtggcc agtgccctgt
481 cctccagata ccactgagct cactgcccat
        gattcagagc tttcaaggat aggctttatt
541 ctgcaagcaa tacaaataat aaatctattc
        tgctgagaga tcac,
```

or a sequence having at least 90% identity to the sequence of SEQ ID NO: 8.

In some embodiments, the composition is stable at an ambient temperature. In some embodiments, the composition is stable at a refrigerated temperature. In some embodiments, the composition is stable above a temperature of at least 4° C. In some embodiments, the composition is stable below a temperature of 30° C.

In some embodiments, endotoxins comprise a cellular lipid, a cellular lipid layer, or a lipopolysaccharide. In some embodiments, the cellular lipid, cellular lipid layer, or lipopolysaccharide is from a human cell. In some embodiments, the cellular lipid, cellular lipid layer, or lipopolysaccharide is from a non-human vertebrate cell. In some embodiments, the cellular lipid, cellular lipid layer, or lipopolysaccharide is from a microbe. In some embodiments, the cellular lipid, a cellular lipid layer and a lipopolysaccharide is from a bacterium.

In some embodiments, the stabilized hemoglobin is non-naturally occurring. In some embodiments, the stabilized hemoglobin is polymerized. In some embodiments, the stabilized hemoglobin has been cross-linked with an aldehyde to form a hemoglobin glutamer. In some embodiments, the aldehyde is glutaraldehyde. In some embodiments, the stabilized hemoglobin has an average molecular weight of 200 kilodaltons (kDa). In some embodiments, the stabilized hemoglobin has an average molecular weight of 68 kDa to 500 kDa. In some embodiments, the stabilized hemoglobin has an average molecular weight of at least 68 kDa. In some embodiments, the stabilized hemoglobin has an average molecular weight of at most 500 kDa. In some embodiments, the stabilized hemoglobin has an average molecular weight of 68 kDa to 100 kDa, 68 kDa to 200 kDa, 68 kDa to 300 kDa, 68 kDa to 400 kDa, 68 kDa to 500 kDa, 100 kDa to 200 kDa, 100 kDa to 300 kDa, 100 kDa to 400 kDa, 100 kDa to 500 kDa, 200 kDa to 300 kDa, 200 kDa to 400 kDa, 200 kDa to 500 kDa, 300 kDa to 400 kDa, 300 kDa to 500 kDa, or 400 kDa to 500 kDa. In some embodiments, the stabilized hemoglobin has an average molecular weight of 68 kDa, 100 kDa, 200 kDa, 300 kDa, 400 kDa, or 500 kDa. In some embodiments, the stabilized hemoglobin has less than 15% molecular weight distribution over 500 kDa. In some embodiments, the stabilized hemoglobin has been substantially deoxygenized prior to stabilization with a stabilizing agent. In some embodiments, the stabilization comprises polymerization. In some embodiments, the stabilization comprises reduction of the stabilizing agent. In some embodiments, the stabilized hemoglobin is concentrated by filtration and/or diafiltration with an electrolyte solution. In some embodiments, the electrolyte solution is a physiologic electrolyte solution. In some embodiments, the filtration is ultrafiltration. In some embodiments, the electrolyte solution minimizes formation of methemoglobin (MetHb). In some embodiments, the electrolyte solution comprises N-acetyl-L-cysteine.

In some embodiments, the composition comprises: (a) less than 10% MetHb, optionally less than 6% MetHb; and/or (b) less than 10% hemoglobin dimers, optionally less than 6% hemoglobin dimers. In some embodiments, the level of MetHb is measured by cooximetry. In some embodiments, the level of hemoglobin dimers is measured by a size separation technique. In some embodiments, the composition comprises at least 20% stabilized active tetrameric hemoglobin, optionally 25% to 35% stabilized active tetrameric hemoglobin. In some embodiments, the composition comprises at least 60% greater-than-tetrameric molecular weight hemoglobin oligomers, optionally at least 70% greater-than-tetrameric molecular weight hemoglobin oligomers. In some embodiments, the stabilized hemoglobin has a longer half-life than non-stabilized or oxygenated hemoglobin and minimizes breakdown of tetrameric hemoglobin into dimers that cause renal toxicity. In some embodiments, the stabilized hemoglobin comprises at least one subunit that is synthesized in vitro. In some embodiments, the at least one subunit comprises a gamma (Y) subunit. In some embodiments, the stabilized hemoglobin is not isolated from a human fetus.

The present disclosure also provides a pharmaceutical formulation comprising a stabilized hemoglobin composition according to any of the foregoing embodiments, wherein the composition further comprises a pharmaceutically-acceptable excipient, a pharmaceutically acceptable solvent or a pharmaceutically-acceptable carrier. In some embodiments, the composition is formulated for intravenous injection. In some embodiments, the composition is formulated for intraosseous injection. The present disclosure additionally provides an injection device comprising a composition according to any of the foregoing embodiments. In another aspect, the disclosure provides an injection device comprising a pharmaceutical formulation according to any of the foregoing embodiments. In some embodiments, the device comprises one or more of a needle, an injection pen, an intravenous (IV) line, a central IV line, a syringe, a catheter, and a blood exchanging and/or filtering device. In some embodiments, the device is intended for administration by an individual who is not a medical professional. In some embodiments, the device comprises a preloaded self-injection device. In some embodiments, the device comprises a preloaded self-injection pen. In some embodiments, the device comprises one or more therapeutically effective doses. In some embodiments, the device comprises one or more unit doses. In some embodiments, the one or more unit doses has a volume of between 10 mL and 30 mL, inclusive of the endpoints. In some embodiments, the one or more unit doses has a volume between 18 mL and 25 mL, inclusive of the endpoints. In some embodiments, the injection device includes a metering device. In some embodiments, the injection device is operably linked to a metering device. In some embodiments, the injection device may be connected to a metering device. In some embodiments, the injection device comprises a titrated dose. In some embodiments, the injection device comprises one or more compartments, each capable of maintaining a preloaded volume of a composition according to any of the foregoing embodiments, and each capable of delivering to a subject a distinct volume of the composition, wherein the volume of the composition in each compartment may be delivered simultaneously or sequentially. In some embodiments, the injection device comprises one or more compartments, each capable of comprising a distinct amount of the formulation buffer to selectively dilute the composition to a predetermined final concentration for each compartment.

In another aspect, the present disclosure provides uses of a composition according to any of the foregoing embodiments for the treatment of a subject in need thereof. The present disclosure also provides uses of a pharmaceutical formulation according to any of the foregoing embodiments for the treatment of a subject in need thereof. The present disclosure also provides uses of an injection device according to any of the foregoing embodiments for the treatment of a subject in need thereof. In some embodiments, the subject is hypoxic and/or anemic. In some embodiments, the subject has experienced blood loss from an injury, blood loss from a medical intervention, hemolysis or reduced hematopoiesis. In some embodiments, the subject is a human. In some embodiments, the subject is a non-human animal. In some embodiments, the non-human animal is a non-human vertebrate, a non-human primate, a cetacean, a mammal, a reptile, a bird, an amphibian, or a fish. In some embodiments, the non-human animal is a bovine or ovine. In some embodiments, the non-human animal is a mustelid, a captive mustelid, a rodent, a captive rodent, a raptor, or a captive bird. In some embodiments, the captive bird is of the order Psittaciformes, Passeriformes or Columbiformes.

Another aspect of the disclosure provides a method of treating comprising administering to a subject in need thereof a composition according to any of the foregoing embodiments. In some embodiments, the administering comprises providing a therapeutically effective amount of the composition to the subject in one or more doses. The present disclosure also provides a method of treating comprising administering to a subject in need thereof a pharmaceutical formulation according to any of the foregoing embodiments. In some embodiments, the administering comprises providing a therapeutically effective amount of the pharmaceutical formulation to the subject in one or more doses. The present disclosure additionally provides a method of treating comprising providing to a subject in need thereof an injection device according to any of the foregoing embodiments, wherein the device injects the composition into the subject, thereby treating the subject. In some embodiments, the injection device comprises a therapeutically effective amount of the composition. In some embodiments, the injection device comprises one or more doses of the composition. In some embodiments, the injection device provides an escalating or de-escalating dosage regime by injecting from each of the one or more compartments, sequentially, (a) an increasing or a decreasing volume of a composition according to any of the foregoing embodiments, respectively, or (b) an increasing or a decreasing concentration of a composition according to any of the foregoing embodiments, respectively. In some embodiments, the injection device provides an escalating or de-escalating dosage regime by injecting from each of the one or more compartments, sequentially, (a) an increasing or a decreasing volume of a pharmaceutical formulation according to any of the foregoing embodiments, respectively, or (b) an increasing or a decreasing concentration of a pharmaceutical formulation according to any of the foregoing embodiments, respectively.

In some embodiments, the subject is hypoxic and/or anemic. In some embodiments, the subject has experienced blood loss from an injury, blood loss from a medical intervention, hemolysis or reduced hematopoiesis. In some embodiments, the subject is a human. In some embodiments, the subject is a non-human animal. In some embodiments, the non-human animal is a non-human vertebrate, a non-human primate, a cetacean, a mammal, a reptile, a bird, an amphibian, or a fish. In some embodiments, the non-human animal is a bovine. In some embodiments, the non-human animal is a mustelid, a captive mustelid, a rodent, a captive rodent, a raptor, or a captive bird. In some embodiments, the captive bird is of the order Psittaciformes, Passeriformes or Columbiformes. In some embodiments, the composition, pharmaceutical formulation, or injection is administered to the subject on a repeated dosing schedule. In some embodiments, the repeated doses are administered to achieve and/or maintain a plasma concentration of 0.3-0.4 g/dL of stabilized hemoglobin.

In one embodiment, the present disclosure provides a hemoglobin based composition comprising less than 0.02 milligrams per milliliter (mg/mL) of dissolved oxygen and a stabilized hemoglobin stabilized by exposure to at least one stabilizing agent, and having 20-35% of the total hemoglobin being in tetrameric form, 15-20% of the total hemoglobin being in octameric form, 40-55% of the total hemoglobin being in greater-than-octameric form, and less than 5% of the total hemoglobin being in dimer form.

In some embodiment, the stabilized hemoglobin is at a concentration of between 70 and 200 grams per Liter (g/L) inclusive of the endpoints, and may be at a concentration of between 150 and 200 g/L inclusive of the endpoints.

In some embodiments, the stabilizing agent is selected from a group consisting of: glutaraldehyde, succindialdehyde, activated forms of polyoxyethylene and dextran, α-hydroxy aldehydes, glycolaldehyde, N-maleimido-6-aminocaproyl-(2′-nitro, 4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl) cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl) butyrate, sulfosuccinimidyl 4-(p-maleimidophenyl)butyrate, 1-ethyl-3-(3-dimethylaminopropyl)carbodiimide hydrochloride, N,N′-phenylene dimaleimide, a bis-imidate compound, an acyl diazide compound, an aryl dihalide compound, or combinations thereof.

In some embodiments, the hemoglobin based composition has a formulation buffer having one or more of borate, anti-oxidants, and electrolytes. In some embodiments, the anti-oxidants include N-acetyl-L-cysteine.

In some embodiments, the hemoglobin based composition has fewer than 0.05 endotoxin units (EU) per milliliter (mL) (EU/mL). In some embodiments, the endotoxins include a cellular lipid, a cellular lipid layer, or a lipopolysaccharide. In various embodiments, the endotoxins may be from a human cell, a non-human vertebrate cell, a microbe, or a bacterium.

In some embodiments, the hemoglobin based composition includes hemoglobin isolated or derived from a human, a human cell, a human cell line, a non-human animal, a non-human cell, or a non-human cell line.

In some embodiments, the composition is stable at a temperature selected from the group consisting of ambient, refrigerated, above a temperature of at least 4° C., or below a temperature of 30° C.

In some embodiments, the stabilized hemoglobin of the hemoglobin based composition is non-naturally occurring.

In some embodiments, the stabilized hemoglobin is polymerized. In some embodiments, the stabilized hemoglobin has been cross-linked with an aldehyde to form a hemoglobin glutamer. In some embodiments, the aldehyde is a glutaraldehyde.

In some embodiments, the hemoglobin based composition provides stabilized hemoglobin having an average molecular weight of 200 kilodaltons (kDa).

In some embodiments, the stabilized hemoglobin has been substantially deoxygenized prior to stabilization with the at least one stabilizing agent. The stabilization may include the process of polymerization. In some embodiments, stabilization further includes the process of reduction of the stabilizing agent.

In some aspects, the stabilized hemoglobin of the hemoglobin based composition includes less than 10% methemoglobin.

In some embodiments, the hemoglobin based composition has at least one subunit of the stabilized hemoglobin that is synthesized in vitro.

In some aspects, the hemoglobin based composition includes a pharmaceutically-acceptable excipient, a pharmaceutically-acceptable solvent or a pharmaceutically-acceptable carrier. In some embodiments, the composition is formulated for intravenous or intraosseous injection.

In some aspects, the hemoglobin based composition has greater than about 80% of the stabilized hemoglobin having a defined molecular weight distribution of between 68 kilodaltons and 500 kilodaltons. In some aspects, the hemoglobin based composition has greater than about 80% of the stabilized hemoglobin having a defined molecular weight distribution of 68 kDa to 500 kDa. In some aspects, the hemoglobin based composition has greater than about 80% of the stabilized hemoglobin having a defined molecular weight distribution of at least 68 kDa. In some aspects, the hemoglobin based composition has greater than about 80% of the stabilized hemoglobin having a defined molecular weight distribution of at most 500 kDa. In some aspects, the hemoglobin based composition has greater than about 80% of the stabilized hemoglobin having a defined molecular weight distribution of 68 kDa to 100 kDa, 68 kDa to 200 kDa, 68 kDa to 300 kDa, 68 kDa to 400 kDa, 68 kDa to 500 kDa, 100 kDa to 200 kDa, 100 kDa to 300 kDa, 100 kDa to 400 kDa, 100 kDa to 500 kDa, 200 kDa to 300 kDa, 200 kDa to 400 kDa, 200 kDa to 500 kDa, 300 kDa to 400 kDa, 300 kDa to 500 kDa, or 400 kDa to 500 kDa. In some aspects, the hemoglobin based composition has greater than about 80% of the stabilized hemoglobin having a defined molecular weight distribution of 68 kDa, 100 kDa, 200 kDa, 300 kDa, 400 kDa, or 500 kDa.

### BRIEF DESCRIPTION OF THE DRAWINGS

FIG. 1 shows the results of an oligomer component analysis of exemplary batches of the stabilized hemoglobin solution as described in Example 2.

FIG. 2 shows an experimental design for the exemplary pharmacokinetic study summarized in Example 4.

FIG. 3 shows an experimental design for the exemplary pharmacokinetic study summarized in Example 5.

### DETAILED DESCRIPTION OF THE INVENTION

The present disclosure relates to stabilized hemoglobin solutions, and the uses of such stabilized hemoglobin solutions in the treatment of a subject in need thereof. The stabilized hemoglobin solution is a monomeric mammalian hemoglobin in cross-linked form, substantially free of endotoxins, phospholipids and non-hemoglobin proteins such as enzymes.

### Definitions

The term “blood substitute” or “hemoglobin-based oxygen carrier” or “HBOC” is intended to be a material having the ability to transport and supply oxygen to vital organs and tissues and to maintain intravascular oncotic pressure. Accordingly, the term encompasses materials known in the art as “plasma expanders” and “resuscitation fluids” as well.

The term “cross-linked” or “polymerized” is intended to encompass both inter-molecular and intramolecular polyhemoglobin, with at least 50% of the polyhemoglobin of greater than tetrameric form.

The terms “deoxygenized” and “deoxygenated” are used interchangeably herein to refer to a hemoglobin composition from which oxygen has been removed, e.g., via diafiltration against a degassing membrane with nitrogen flowing across the opposite side of the membrane. A composition that has been “substantially deoxygenized” as used herein refers to a composition comprising less than 0.02 milligrams (mg) per milliliter (mL) (mg/mL) of dissolved oxygen.

By the term “endotoxin(s)” is intended the generally cell-bound lipopolysaccharides produced as a part of the outer layer of bacterial cell walls, which under many conditions are toxic. When injected into an animal, endotoxins cause fever, diarrhea, hemorrhagic shock, and other tissue damage.

By the term “endotoxin unit” (EU) is intended that meaning given by the United States Pharmacopeial Convention of 1983, Page 3014, which defined EU as the activity contained in 0.2 nanograms of the U.S. reference standard lot EC-2. One vial of EC-2 contains 5,000 EU.

“Hemoglobin” or “Hb” is the protein molecule in red blood cells that carries oxygen from the lungs to the body's tissues and returns carbon dioxide from the tissues back to the lungs. Hemoglobin is typically composed of four globulin chains. The normal adult hemoglobin molecule contains two alpha-globulin chains and two beta-globulin chains. In fetuses and infants, beta chains are not common and the hemoglobin molecule is made up of two alpha chains and two gamma chains. Each globulin chain contains an important iron-containing porphyrin compound termed heme. Embedded within the heme compound is an iron atom that is vital in transporting oxygen and carbon dioxide in our blood. The iron contained in hemoglobin is also responsible for the red color of blood.

A “glutamer” or “hemoglobin glutamer” as referenced herein refers to a blood substitute or hemoglobin-based oxygen carrier as described in the “International Nonproprietary Names for Pharmaceutical Substances (INN)”, WHO Drug Information. Additional generic names for such substances include Hemoglobin Glutamer-200, HBOC-301, Hemoglobin Glutamer-250, and HBOC-201.

“Methemoglobin” or “methaemoglobin” is a hemoglobin in the form of metalloprotein, in which the iron in the heme group is in the Fe3+(ferric) state, not the Fe2+(ferrous) of normal hemoglobin. Methemoglobin cannot bind oxygen, which means it cannot carry oxygen to tissues. In human blood, a trace amount of methemoglobin is normally produced spontaneously, but when present in excess the blood becomes abnormally dark bluish brown. The NADH-dependent enzyme methemoglobin reductase (a type of diaphorase) is responsible for converting methemoglobin back to hemoglobin. Normally one to two percent of a person's hemoglobin is methemoglobin; a higher percentage than this can be genetic or caused by exposure to various chemicals and depending on the level can cause health problems known as methemoglobinemia. An abnormal increase of methemoglobin will increase the oxygen binding affinity of normal hemoglobin, resulting in a decreased unloading of oxygen to the tissues and possible tissue hypoxia.

“Oxyhemoglobin” or “oxyhaemoglobin” is the oxygen-loaded form of hemoglobin. In general, hemoglobin can be saturated with oxygen molecules (oxyhemoglobin), or desaturated with oxygen molecules (deoxyhemoglobin). Oxyhemoglobin is formed during physiological respiration when oxygen binds to the heme component of hemoglobin in red blood cells. This process occurs in the pulmonary capillaries adjacent to the alveoli of the lungs. The oxygen then travels through the blood stream to be dropped off at cells where it is utilized as a terminal electron acceptor in the production of ATP by the process of oxidative phosphorylation.

The terms “stabilized hemoglobin solution” and “stabilized hemoglobin composition” refer to the disclosed compositions comprising cross-linked (i.e., stabilized) deoxygenated hemoglobin. Such solutions may be prepared in a pharmaceutical formulation and/or provided in an injection device and may be used to treat one or more anemic or hypoxic conditions.

As used herein, the term “stabilized active tetrameric hemoglobin” refers to stabilized, e.g., cross-linked, tetrameric hemoglobin comprising linked alpha-beta and alpha-beta sub chains.

As used herein, “a stabilizing agent” refers to any agent that may be used to stabilize, polymerize, or cross-link the hemoglobin oligomers comprised by a hemoglobin composition according to the present disclosure. Exemplary stabilizing agents include, for example, aldehydes. Suitable stabilizing agents include, as non-limiting examples, glutaraldehyde, succindialdehyde, activated forms of polyoxyethylene and dextran, α-hydroxy aldehydes, such as glycolaldehyde, N-maleimido-6-aminocaproyl-(2′-nitro, 4′-sulfonic acid)-phenyl ester, m-maleimidobenzoic acid-N-hydroxysuccinimide ester, succinimidyl 4-(N-maleimidomethyl)cyclohexane-1-carboxy late, sulfosuccinimidyl 4-(N-maleimidomethyl) cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl) butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bis-imidate class, the acyl diazide class or the aryl dihalide class, among others.

The term “substantially endotoxin free”, for the purposes of the present invention, may be described functionally as a stabilized hemoglobin composition which contains less than 1.0 endotoxin units per milliliter of solution, at a concentration of 10 grams of hemoglobin per deciliter of solution, though the final concentration may be between 15 and 20 grams of hemoglobin per deciliter of solution. In some embodiments, the “substantially endotoxin free” hemoglobin drug substance of the present disclosure will contain less than 0.5, and preferably less than 0.25, most preferably less than 0.02 endotoxin units per milliliter of solution (EU/ml) as measured by the Limulus Amebocytic Lysate (LAL) assay. The LAL assay is described by Nachum et al., Laboratory Medicine, 13:112-117 (1982) and Pearson III et al., Bioscience, 30:416-464 (1980), incorporated by reference herein.

### Stabilized Hemoglobin Compositions and Formulation Thereof

The present disclosure relates to stabilized hemoglobin solutions. Without wishing to be bound by theory, it is theorized that existing hemoglobin-based oxygen carriers and blood substitutes have elicited undesirable and sometimes dangerous side effects in part due to the high oxygen content of the hemoglobin comprised by such solutions. However, it has historically proven difficult to obtain highly concentrated deoxygenated hemoglobin solutions. The present application is the first to disclose highly concentrated, predominantly deoxygenated, stabilized hemoglobin solutions. The following subsections provide exemplary methods for obtaining and producing the stabilized hemoglobin solutions of the present disclosure and provide exemplary characteristics of the resulting solutions. Additional features useful in the present disclosure may be found in International Publication No. WO 2019/055489, the contents of which are herein incorporated by reference in their entirety.

Hemoglobin and/or Red Blood Cell Source

The hemoglobin comprised in the stabilized hemoglobin compositions of the present disclosure may be obtained from an organism or may be synthetically formulated.

In some embodiments, the hemoglobin is obtained from an erythrocyte (red blood cell) source. In some embodiments, the hemoglobin is derived from a human source. In some embodiments, the hemoglobin comprises hemoglobin isolated or derived from a human, a human cell, or a human cell line. In some embodiments, the red blood cells may be from freshly drawn human blood, expired blood from blood banks (i.e., donated blood that has exceeded its shelf life), placentas, or packed erythrocytes obtained from human donor centers. In some embodiments, the hemoglobin is derived from human cells more than 15 days after harvest. In some embodiments, the hemoglobin is derived from fewer than 100 variable sources. In some embodiments, the hemoglobin is derived from fewer than 200, fewer than 100, fewer than 90, fewer than 80, fewer than 70, fewer than 60, fewer than 50, fewer than 40, fewer than 30, fewer than 20, or fewer than 10 variable sources. In some embodiments, the stabilized hemoglobin is not isolated from a human fetus.

In some embodiments, the stabilized hemoglobin solution comprises hemoglobin isolated or derived from a non-human animal, a non-human cell or a non-human cell line. In some embodiments, the hemoglobin is derived from a fresh source of red blood cells. In some embodiments, the hemoglobin is isolated or derived from cells fewer than ten days after harvest. In some embodiments, stabilized hemoglobin solutions may comprise hemoglobin derived or isolated from a non-human animal that is a non-human vertebrate, a non-human primate, a cetacean, a mammal, a reptile, a bird, an amphibian, or a fish. In some embodiments, red blood cells obtained from animal blood are used. In some embodiments, the hemoglobin is derived from a nonhuman mammalian blood source. Blood from a variety of sources such as bovine, ovine, or porcine may be used. In some embodiments, ovine blood may be used. Because of its ready availability, in some embodiments, bovine blood may be used. In some embodiments, the hemoglobin is derived from a bovine blood source.

In some embodiments, stabilized hemoglobin solutions may comprise hemoglobin derived or isolated from a non-human animal that is a mustelid, a captive mustelid, a rodent, a captive rodent, a raptor, or a captive bird. In some embodiments, the captive bird is of the order Psittaciformes, Passeriformes, or Columbiformes. In some embodiments, the non-human animal is not a squab that is raised for food.

In some embodiments, the stabilized hemoglobin solution may comprise hemoglobin that is partially or wholly synthetic. In some embodiments, the stabilized hemoglobin solution may comprise at least one subunit that is synthesized in vitro. In some embodiments, the stabilized hemoglobin solution may comprise at least one synthetic subunit comprising a gamma (Y) subunit.

### Red Blood Cell Collection

In some embodiments, the present stabilized hemoglobin solutions may comprise hemoglobin that is derived or isolated from red blood cells collected from a non-human animal source. For collection of red blood cells from, e.g., bovine sources, collection trochars may be used to extract the blood in a sterile manner. The trochars are carefully inserted and handled and are connected to tubing approximately 2 feet in length. In order to insert the trochar, the hide is cut away and peeled back, and the trochar is then inserted in the animal's major vessels close to the heart with care not to puncture the esophagus. Avoiding the introduction of bacteria and the maintenance of endotoxin-free of low endotoxin level material is important. This may be accomplished using individual containers that are pre-charged with an anticoagulant and that are depyrogenated and re-checked for endotoxins. Typical anticoagulants include sodium citrate. In all cases, endotoxin levels of the containers must be less than 0.01 endotoxin units as detected by LAL.

During or after collection, the collected blood may be treated so as to prevent coagulation. In some embodiments, the collecting vessel may be treated with an anticoagulant. In some embodiments, the collected blood may be defibrinated or citrated. Defibrinated blood is blood from which fibrin has been removed or which has been treated to denature fibrinogen without causing cell lysis. Citrated blood is blood that has been treated with sodium citrate or citric acid to prevent coagulation.

The red blood cell solution may be distributed to small vessels that can hold between 2 to 10 gallons of gathered blood in a sterile manner and, therefore, maintain the blood in an endotoxin-free state. The collected blood in its container may be capped off immediately to avoid exposure to the environment. Upon completion of the collection process, the material is chilled, typically to about 4° C., to limit bacterial growth. There is no pooling of blood at this time; the blood is later checked for endotoxins and sterility to ensure that (1) no one cow is sick; or (2) a bad collection technique has not contaminated the entire batch or collection for that day.

The illustrative collection method described in the foregoing section is not meant to be limiting, as there are many collection methods which are suitable and available to one with ordinary skill in the art.

### Red Blood Cell Processing and Stabilized Hemoglobin Compositions Formulation

An additional aspect of the present disclosure describes the process by which the stabilized hemoglobin compositions are prepared. An exemplary method for the processing of red blood cells and formulation of the stabilized hemoglobin compositions according to the present disclosure is provided in Example 1 infra.

Generally, in some embodiments, the stabilized hemoglobin composition is prepared from a mammalian blood fraction by a process comprising 1) separation of red blood cells from the mammalian blood fraction; 2) hemolysis of the red blood cells to produce a composite of monomeric hemoglobin and stroma; 3) separation by filtration of the hemoglobin; 4) purification of the monomeric hemoglobin by high performance liquid chromatography (HPLC) to separate the hemoglobin from all other proteins residual of the red blood cells, as well as the phospholipid, enzyme and endotoxin contaminants; 5) deoxygenation and diafiltration; 6) crosslinking (polymerizing or aggregating) the monomeric hemoglobin; and/or 7) concentrating the stabilized hemoglobin solution.

In some embodiments, the process may comprise the steps of (1) obtaining the blood raw product, (2) fractionating the blood raw product to produce a red blood cell fraction which is substantially free from white blood cells and platelets, (3) mechanically disrupting the red blood cell fraction to produce a hemoglobin-containing solution, (4) clarifying the hemoglobin-containing solution to produce a hemoglobin solution which is substantially free of cellular debris, (5) microporously filtering the hemoglobin solution which is substantially free of cellular debris to produce a partially sterilized hemoglobin-containing solution, (6) ultrafiltering the partially sterilized hemoglobin-containing solution to produce a size-separated hemoglobin-containing solution, (7) chromatographically separating the size-separated hemoglobin-containing solution to produce a hemoglobin substantially free of phospholipids, nonhemoglobin proteins, and endotoxins, (8) deoxygenating the substantially endotoxin-free hemoglobin to produce a substantially deoxygenated hemoglobin solution, (9) cross-linking said substantially deoxygenated hemoglobin solution to produce stabilized hemoglobin solution, and/or (10) concentrating the stabilized hemoglobin solution, all steps done in a substantially endotoxin-free environment.

In some embodiments, stabilizing the hemoglobin solution comprises polymerizing the hemoglobin solution, e.g., through cross-linking. Any cross-linking agent known in the art may be employed. In some embodiments, the cross-linking agent is an aldehyde. In some embodiments, the aldehyde is a glutaraldehyde. In some embodiments, the process may comprise a step after the cross-linking step to separate or partially separate monomeric and low molecular weight species of hemoglobin from the higher molecular weight polymers formed during crosslinking. In some embodiments, the process also comprises a step of concentrating the stabilized, deoxygenated hemoglobin solution to a concentration between 70 g/L-200 g/L, and may be desirably concentrated to suitable ranges including: 70 g/L-100 g/L, 85 g/L-125 g/L, 95 g/L-150 g/L, or 150 g/L to about 200 g/L (inclusive of end points) of hemoglobin in solution.

In some embodiments, the process may comprise the addition of in vitro synthesized hemoglobin at any stage prior to cross-linking. In some embodiments, the process comprises formulating highly concentrated, deoxygenated, stabilized hemoglobin from a synthetic source.

In some embodiments, the process may comprise conducting any one or more of the above steps under conditions which result in a product which is substantially free of endotoxins, phospholipids and non-hemoglobin proteins such as enzymes, and has a defined molecular weight distribution of greater than about 90% between 68,000 daltons and 500,000 daltons.

In some embodiments, the process may be conducted in a substantially endotoxin-free environment, such that the endotoxin reading does not exceed 0.05 EU/mL at any stage of the manufacturing process.

### Characteristics of Stabilized Hemoglobin Solutions

Stabilized hemoglobin solutions according to the present disclosure may have one or more characteristics that make them particularly suitable for in vitro, in vivo, experimental, and/or therapeutic applications. In some embodiments, the stabilized hemoglobin solutions may have one or more of the following attributes: high hemoglobin concentration, low dissolved oxygen concentration, low endotoxin concentration, long half-life, high average molecular weight, and a high percentage of greater-than-dimeric polymers of hemoglobin.

In some embodiments, a stabilized hemoglobin solution according to the present disclosure may have a higher concentration than other hemoglobin-based oxygen carriers or hemoglobin-based blood substitutes that are commercially available or under clinical review. In some embodiments, a stabilized hemoglobin solution of the present disclosure may have a concentration of about 70 g/L-200 g/L, and may be desirably concentrated to suitable ranges including: 70 g/L-100 g/L, 85 g/L-125 g/L, 95 g/L-150 g/L, or 150 g/L to about 200 g/L. In some embodiments, a stabilized hemoglobin solution of the present disclosure may have a concentration of at least about 150 g/L. In some embodiments, a stabilized hemoglobin solution of the present disclosure may have a concentration of at most about 200 g/L. In some embodiments, a stabilized hemoglobin solution of the present disclosure may have a concentration of about 150 g/L to about 155 g/L, about 150 g/L to about 160 g/L, about 150) g/L to about 165 g/L, about 150 g/L to about 170 g/L, about 150 g/L to about 175 g/L, about 150 g/L to about 180 g/L, about 150 g/L to about 185 g/L, about 150 g/L to about 190 g/L, about 150 g/L to about 195 g/L, about 150 g/L to about 200 g/L, about 155 g/L to about 160 g/L, about 155 g/L to about 165 g/L, about 155 g/L to about 170 g/L, about 155 g/L to about 175 g/L, about 155 g/L to about 180 g/L, about 155 g/L to about 185 g/L, about 155 g/L to about 190 g/L, about 155 g/L to about 195 g/L, about 155 g/L to about 200 g/L, about 160 g/L to about 165 g/L, about 160 g/L to about 170 g/L, about 160 g/L to about 175 g/L, about 160 g/L to about 180 g/L, about 160 g/L to about 185 g/L, about 160 g/L to about 190 g/L, about 160 g/L to about 195 g/L, about 160 g/L to about 200 g/L, about 165 g/L to about 170 g/L, about 165 g/L to about 175 g/L, about 165 g/L to about 180 g/L, about 165 g/L to about 185 g/L, about 165 g/L to about 190 g/L, about 165 g/L to about 195 g/L, about 165 g/L to about 200 g/L, about 170 g/L to about 175 g/L, about 170 g/L to about 180 g/L, about 170 g/L to about 185 g/L, about 170 g/L to about 190 g/L, about 170 g/L to about 195 g/L, about 170 g/L to about 200 g/L, about 175 g/L to about 180 g/L, about 175 g/L to about 185 g/L, about 175 g/L to about 190 g/L, about 175 g/L to about 195 g/L, about 175 g/L to about 200 g/L, about 180 g/L to about 185 g/L, about 180 g/L to about 190 g/L, about 180 g/L to about 195 g/L, about 180 g/L to about 200 g/L, about 185 g/L to about 190 g/L, about 185 g/L to about 195 g/L, about 185 g/L to about 200 g/L, about 190 g/L to about 195 g/L, about 190 g/L to about 200 g/L, or about 195 g/L to about 200 g/L. In some embodiments, a stabilized hemoglobin solution of the present disclosure may have a concentration of about 150 g/L, about 155 g/L, about 160 g/L, about 165 g/L, about 170 g/L, about 175 g/L, about 180 g/L, about 185 g/L, about 190 g/L, about 195 g/L, or about 200 g/L.

In some embodiments, a stabilized hemoglobin solution of the present disclosure may have a lower oxygen concentration than other hemoglobin-based oxygen carriers or hemoglobin based blood substitutes that are commercially available or under clinical review. In some embodiments, the dissolved oxygen concentration is less than 0.1 mg/mL, less than 0.09 mg/mL, less than 0.08 mg/mL, less than 0.07 mg/mL, less than 0.06 mg/mL, less than 0.05 mg/mL, less than 0.04 mg/mL, less than 0.03 mg/mL, less than 0.02 mg/mL, or less than 0.01 mg/mL. In some embodiments, the dissolved oxygen concentration is less than 0.02 mg/mL. In some embodiments, the stabilized hemoglobin solution comprises less than 5% oxygenated hemoglobin as a percentage of overall hemoglobin. In some embodiments, the stabilized hemoglobin solution comprises less than 10%, less than 9%, less than 8%, less than 7%, less than 6%, less than 5%, less than 4%, less than 3%, or less than 2% oxygenated hemoglobin as a percentage of overall hemoglobin. In some embodiments, the stabilized hemoglobin solution comprises less than 3% oxygenated hemoglobin as a percentage of overall hemoglobin.

In some embodiments, the stabilized hemoglobin solution may contain little to no endotoxin contamination. In some embodiments, the stabilized hemoglobin solution is substantially free of endotoxins, phospholipids and non-hemoglobin proteins such as enzymes. In some embodiments, the stabilized hemoglobin solution may be virtually free of endotoxins. In some embodiments, the endotoxin concentration of a stabilized hemoglobin solution according to the present disclosure may be less than about 0.05 endotoxin units (EU) per milliliter (mL). In some embodiments, the endotoxin concentration of a stabilized hemoglobin solution according to the present disclosure may be less than about 0.5, 0.4, 0.3, 0.2, 0.1, 0.05, 0.04, 0.03, 0.02, or 0.01 EU per mL. In some embodiments, the measured endotoxins may comprise one or more of a cellular lipid, a cellular lipid layer and a lipopolysaccharide. In some embodiments, the endotoxin may be derived or isolated from a human cell. In some embodiments, the endotoxin may be derived or isolated from a non-human vertebrate cell. In some embodiments, the endotoxin may be derived or isolated from a microbe. In some embodiments, the endotoxin may be derived or isolated from a bacterium. In some embodiments, the endotoxin may be derived or isolated from a virus.

In some embodiments, the stabilized hemoglobin solution may comprise a distribution of hemoglobin oligomers of different sizes. In some embodiments, the stabilized hemoglobin solution may comprise virtually no hemoglobin monomers. In some embodiments, the stabilized hemoglobin solution may comprise less than 15%, less than 10%, less than 9%, less than 8%, less than 7%, less than 6%, or less than 5% hemoglobin dimers. In some embodiments, the stabilized hemoglobin solution may comprise less than 10% hemoglobin dimers. In some embodiments, the composition may comprise less than 6% hemoglobin dimers. The level of hemoglobin dimers may be measured by known methods. In some embodiments, the level of hemoglobin dimers comprised by a solution is measured via size separation techniques, e.g., chromatography or SDS-PAGE. In some embodiments, the stabilized hemoglobin solution may comprise greater than 80%, greater than 85%, or greater than 90% hemoglobin oligomers between 68,000 daltons and 500,000 daltons. In some embodiments, the stabilized hemoglobin solution may comprise between 20% to 35% hemoglobin tetramers. In some embodiments, the stabilized hemoglobin solution may comprise about 20%, about 21%, about 22%, about 23%, about 24%, about 25%, about 26%, about 27%, about 28%, about 29%, about 30%, about 31%, about 32%, about 33%, about 34%, or about 35% hemoglobin tetramers. In some embodiments, the stabilized hemoglobin solution may comprise about 25% hemoglobin tetramers. In some embodiments, the hemoglobin solution may comprise between 15% and 25% hemoglobin octamers. In some embodiment, the hemoglobin solution may comprise between 15% and 20% hemoglobin octamers. In some embodiments, the stabilized hemoglobin solution may comprise about 15%, about 16%, about 17%, about 18%, about 19%, about 20%, about 21%, about 22%, about 23%, about 24%, or about 25% hemoglobin octamers. In some embodiments, the stabilized hemoglobin solution may comprise about 20% hemoglobin octamers. In some embodiments, the stabilized hemoglobin solution may comprise between 40% and 55% hemoglobin oligomers of greater-than-octamer size. In some embodiments, the stabilized hemoglobin solution may comprise about 40%, about 41%, about 42%, about 43%, about 44%, about 45%, about 46%, about 47%, about 48%, about 49%, about 50%, about 51%, about 52%, about 53%, about 54%, or about 55% hemoglobin oligomers of greater-than-octamer molecular weight. In some embodiments, the stabilized hemoglobin solution comprises about 50% hemoglobin oligomers of greater-than-octamer molecular weight.

In some embodiments, the stabilized hemoglobin solution comprises hemoglobin oligomers with a defined molecular weight distribution of greater than about 90% between 68,000 daltons and 500,000 daltons. In some embodiments, the stabilized hemoglobin solution comprises hemoglobin oligomers with a defined molecular weight distribution of greater than about 80% between 68,000 daltons and 500,000 daltons. In some embodiments, the stabilized hemoglobin solution may comprise hemoglobin oligomers having an average molecular weight of 200 kilodaltons (kDa). In some embodiments, the stabilized hemoglobin solution may have a molecular weight distribution comprising less than 15% oligomers over 500 kDa in size. In some embodiments, the stabilized hemoglobin solution may have a molecular weight distribution comprising less than 20%, less than 19%, less than 18%, less than 17%, less than 16%, less than 15%, less than 14%, less than 13%, less than 12%, less than 11%, less than 10%, less than 9%, less than 8%, or less than 7% oligomers over 500 kDa in size.

The existence of methemoglobin may reduce the ability of the hemoglobin solution to release oxygen. In some embodiments, the stabilized hemoglobin solution comprises less than 10% methemoglobin as a percentage of overall hemoglobin. In some embodiments, the stabilized hemoglobin solution comprises less than 10%, less than 9%, less than 8%, less than 7%, less than 6%, less than 5%, less than 4%, less than 3%, less than 2%, less than 1%, or less than 0.5% methemoglobin as a percentage of overall hemoglobin. In some embodiments, the stabilized hemoglobin solution comprises less than 6% methemoglobin as a percentage of overall hemoglobin. In some embodiments, the stabilized hemoglobin solution comprises less than about 1% methemoglobin as a percentage of overall hemoglobin. The level of methemoglobin may be measured according to methods known in the art. In some embodiments, the level of methemoglobin is measured by cooximetry.

In some embodiments, the stabilized hemoglobin has a longer half-life than nonstabilized or oxygenated hemoglobin and minimizes breakdown of tetrameric hemoglobin into dimers that cause renal toxicity. In some embodiments, the stabilized hemoglobin has a half-life of at least 60 minutes, at least 90 minutes, at least 120 minutes, at least 150 minutes, at least 180 minutes, at least 210 minutes, or at least 240) minutes. In some embodiments, the stabilized hemoglobin has a half-life of about 3.5 hours or about 210 minutes.

In some embodiments, the stabilized hemoglobin composition may be stable at various temperatures. In some embodiments, the stabilized hemoglobin is stable at ambient temperature. In some embodiments, the stabilized hemoglobin is stable at refrigerated temperatures. In some embodiments, the stabilized hemoglobin is stable at temperatures above approximately 4° C. In some embodiments, the stabilized hemoglobin is stable at temperatures above 1° C., above 2° C., above 3° C., above 4° C., or above 5° C. In some embodiments, the stabilized hemoglobin is stable at temperatures below approximately 30° C. In some embodiments, the stabilized hemoglobin is stable at temperatures below 35° C., below 34° C., below 33° C., below 32° C., below 31° C., or below 30° C.

### Hemoglobin Sequences

In some embodiments, the hemoglobin comprised by the present stabilized hemoglobin solutions comprises a subunit alpha (a), wherein the subunit α comprises the amino acid sequence of:

```
(SEQ ID NO: 1)
    1 MVLSPADKTN VKAAWGKVGA HAGEYGAEAL
        ERMFLSFPTT KTYFPHFDLS HGSAQVKGHG
  61 KKVADALTNA VAHVDDMPNA LSALSDLHAH
        KLRVDPVNFK LLSHCLLVTL AAHLPAEFTP
121 AVHASLDKFL ASVSTVLTSK YR.
```

In some embodiments, the hemoglobin comprises a subunit α comprising an amino acid sequence having at least 50%, at least 60%, at least 70%, at least 80%, at least 90%, at least 95%, at least 98%, or at least 99% identity to the sequence of SEQ ID NO: 1. In some embodiments, the hemoglobin comprises a subunit α comprising an amino acid sequence having at least 90% identity to the sequence of SEQ ID NO: 1.

In some embodiments, the hemoglobin comprises a subunit α, wherein the subunit α is encoded by the nucleic acid sequence of:

```
(SEQ ID NO: 2)
    1 actcttctgg tccccacaga ctcagagaga
        acccaccatg gtgctgtctc ctgccgacaa
  61 gaccaacgtc aaggccgcct ggggcaaggt
        tggcgcgcac gctggcgagt atggtgcgga
121 ggccctggag aggatgttcc tgtccttccc
        caccaccaag acctacttcc cgcacttcga
181 cctgagccac ggctctgccc aggttaaggg
        ccacggcaag aaggtggccg acgcgctgac
241 caacgccgtg gcgcacgtgg acgacatgcc
        caacgcgctg tccgccctga gcgacctgca
301 cgcgcacaag cttcgggtgg acccggtcaa
        cttcaagctc ctaagccact gcctgctggt
361 gaccctggcc gcccacctcc ccgccgagtt
        cacccctgcg gtgcacgcct ccctggacaa
421 gttcctggct tctgtgagca ccgtgctgac
        ctccaaatac cgttaagctg gagcctcggt
481 agcagttcct cctgccagat gggcctccca
        acgggccctc ctcccctcct tgcaccggcc
541 cttcctggtc tttgaataaa gtctgagtgg
        gcggc.
```

In some embodiments, the hemoglobin comprises a subunit α, wherein the subunit α is encoded by a nucleic acid sequence having at least 50%, at least 60%, at least 70%, at least 80%, at least 90%, at least 95%, at least 98%, or at least 99% identity to the sequence of SEQ ID NO: 2. In some embodiments, the hemoglobin comprises a subunit α, wherein the subunit α is encoded by a nucleic acid sequence having at least 90% identity to the sequence of SEQ ID NO: 2.

In some embodiments, the hemoglobin comprises a subunit beta (B), wherein the subunit β comprises the amino acid sequence of:

```
(SEQ ID NO: 3)
    1 MVHLTPEEKS AVTALWGKVN VDEVGGEALG
        RLLVVYPWTQ RFFESFGDLS TPDAVMGNPK
  61 VKAHGKKVLG AFSDGLAHLD NLKGTFATLS
        ELHCDKLHVD PENFRLLGNV LVCVLAHHFG
121 KEFTPPVQAA YQKVVAGVAN ALAHKYH
```

In some embodiments, the hemoglobin comprises a subunit β, wherein the subunit β comprises an amino acid sequence having at least 50%, at least 60%, at least 70%, at least 80%, at least 90%, at least 95%, at least 98%, or at least 99% identity to the sequence of SEQ ID NO: 3. In some embodiments, the hemoglobin comprises a subunit β, wherein the subunit β comprises an amino acid sequence having at least 90% identity to the sequence of SEQ ID NO: 3.

In some embodiments, the hemoglobin comprises a subunit β, wherein the subunit β is encoded by the nucleic acid sequence of:

```
(SEQ ID NO: 4)
    1 acatttgctt ctgacacaac tgtgttcact
        agcaacctca aacagacacc atggtgcatc
  61 tgactcctga ggagaagtct gccgttactg
        ccctgtgggg caaggtgaac gtggatgaag
121 ttggtggtga ggccctgggc aggctgctgg
        tggtctaccc ttggacccag aggttctttg
181 agtcctttgg ggatctgtcc actcctgatg
        ctgttatggg caaccctaag gtgaaggctc
241 atggcaagaa agtgctcggt gcctttagtg
        atggcctggc tcacctggac aacctcaagg
301 gcacctttgc cacactgagt gagctgcact
        gtgacaagct gcacgtggat cctgagaact
361 tcaggctcct gggcaacgtg ctggtctgtg
        tgctggccca tcactttggc aaagaattca
421 ccccaccagt gcaggctgcc tatcagaaag
        tggtggctgg tgtggctaat gccctggccc
481 acaagtatca ctaagctcgc tttcttgctg
        tccaatttct attaaaggtt cctttgttcc
541 ctaagtccaa ctactaaact gggggatatt
        atgaagggcc ttgagcatct ggattctgcc
601 taataaaaaa catttatttt cattgcaa.
```

In some embodiments, the hemoglobin comprises a subunit β, wherein the subunit β is encoded by a nucleic acid sequence having at least 50%, at least 60%, at least 70%, at least 80%, at least 90%, at least 95%, at least 98%, or at least 99% identity to the sequence of SEQ ID NO: 4. In some embodiments, the hemoglobin comprises a subunit β, wherein the subunit β is encoded by a nucleic acid sequence having at least 90% identity to the sequence of SEQ ID NO: 4.

In some embodiments, the hemoglobin comprises a subunit gamma (γ), wherein the subunit γ comprises the amino acid sequence of:

```
(SEQ ID NO: 5)
    1 MGHFTEEDKA TITSLWGKVN VEDAGGETLG
        RLLVVYPWTQ RFFDSFGNLS SASAIMGNPK
  61 VKAHGKKVLT SLGDAIKHLD DLKGTFAQLS
        ELHCDKLHVD PENFKLLGNV LVTVLAIHFG
121 KEFTPEVQAS WQKMVTGVAS ALSSRYH,
```

In some embodiments, the hemoglobin comprises a subunit γ, wherein the subunit γ comprises an amino acid sequence having at least 50%, at least 60%, at least 70%, at least 80%, at least 90%, at least 95%, at least 98%, or at least 99% identity to the sequence of SEQ ID NO: 5. In some embodiments, the hemoglobin comprises a subunit γ, wherein the subunit γ comprises an amino acid sequence having at least 90% identity to the sequence of SEQ ID NO: 5.

In some embodiments, the hemoglobin comprises a subunit γ, wherein the subunit γ is encoded by the nucleic acid sequence of:

```
(SEQ ID NO: 6)
    1 acactcgctt ctggaacgtc tgaggttatc
        aataagctcc tagtccagac gccatgggtc
  61 atttcacaga ggaggacaag gctactatca
        caagcctgtg gggcaaggtg aatgtggaag
121 atgctggagg agaaaccctg ggaaggctcc
        tggttgtcta cccatggacc cagaggttct
181 ttgacagctt tggcaacctg tcctctgcct
        ctgccatcat gggcaacccc aaagtcaagg
241 cacatggcaa gaaggtgctg acttccttgg
        gagatgccat aaagcacctg gatgatctca
301 agggcacctt tgcccagctg agtgaactgc
        actgtgacaa gctgcatgtg gatcctgaga
361 acttcaagct cctgggaaat gtgctggtga
        ccgttttggc aatccatttc ggcaaagaat
421 tcacccctga ggtgcaggct tcctggcaga
        agatggtgac tggagtggcc agtgccctgt
481 cctccagata ccactgagct cactgcccat
        gatgcagagc tttcaaggat aggctttatt
541 ctgcaagcaa tcaaataata aatctattct
        gctaagagat cacaca,
```

In some embodiments, the hemoglobin comprises a subunit γ, wherein the subunit γ is encoded by a nucleic acid sequence having at least 50%, at least 60%, at least 70%, at least 80%, at least 90%, at least 95%, at least 98%, or at least 99% identity to the sequence of SEQ ID NO: 6. In some embodiments, the hemoglobin comprises a subunit γ, wherein the subunit γ is encoded by a nucleic acid sequence having at least 90% identity to the sequence of SEQ ID NO: 6.

In some embodiments, the hemoglobin comprises a subunit gamma (γ), wherein the subunit γ comprises the amino acid sequence of:

```
(SEQ ID NO: 7)
    1 MGHFTEEDKA TITSLWGKVN VEDAGGETLG
        RLLVVYPWTQ RFFDSFGNLS SASAIMGNPK
  61 VKAHGKKVLT SLGDATKHLD DLKGTFAQLS
        ELHCDKLHVD PENFKLLGNV LVTVLAIHFG
121 KEFTPEVQAS WQKMVTAVAS ALSSRYH
```

In some embodiments, the hemoglobin comprises a subunit γ, wherein the subunit γ comprises an amino acid sequence having at least 50%, at least 60%, at least 70%, at least 80%, at least 90%, at least 95%, at least 98%, or at least 99% identity to the sequence of SEQ ID NO: 7. In some embodiments, the hemoglobin comprises a subunit γ, wherein the subunit γ comprises an amino acid sequence having at least 90% identity to the sequence of SEQ ID NO: 7.

In some embodiments, the hemoglobin comprises a subunit γ, wherein the subunit γ is encoded by the nucleic acid sequence of:

```
(SEQ ID NO: 8)
    1 acactcgctt ctggaacgtc tgaggttatc
        aataagctcc tagtccagac gccatgggtc
  61 atttcacaga ggaggacaag gctactatca
        caagcctgtg gggcaaggtg aatgtggaag
121 atgctggagg agaaaccctg ggaaggctcc
        tggttgtcta cccatggacc cagaggttct
181 ttgacagctt tggcaacctg tcctctgcct
        ctgccatcat gggcaacccc aaagtcaagg
241 cacatggcaa gaaggtgctg acttccttgg
        gagatgccac aaagcacctg gatgatctca
301 agggcacctt tgcccagctg agtgaactgc
        actgtgacaa gctgcatgtg gatcctgaga
361 acttcaagct cctgggaaat gtgctggtga
        ccgttttggc aatccatttc ggcaaagaat
421 tcacccctga ggtgcaggct tcctggcaga
        agatggtgac tgcagtggcc agtgccctgt
481 cctccagata ccactgagct cactgcccat
        gattcagagc tttcaaggat aggctttatt
541 ctgcaagcaa tacaaataat aaatctattc
        tgctgagaga tcac
```

In some embodiments, the hemoglobin comprises a subunit γ, wherein the subunit γ is encoded by a nucleic acid sequence having at least 50%, at least 60%, at least 70%, at least 80%, at least 90%, at least 95%, at least 98%, or at least 99% identity to the sequence of SEQ ID NO: 8. In some embodiments, the hemoglobin comprises a subunit γ, wherein the subunit γ is encoded by a nucleic acid sequence having at least 90% identity to the sequence of SEQ ID NO: 8.

### Pharmaceutical Formulations

Another aspect of the present disclosure relates to pharmaceutical formulations comprising the stabilized hemoglobin solution of the disclosure. Pharmaceutical formulations described herein can be prepared by any method known in the art of pharmacology. In general, such preparatory methods include the steps of bringing the stabilized hemoglobin solution into association with the excipient and/or one or more other accessory ingredients, and then, if necessary and/or desirable, packaging the product into a desired single- or multi-dose unit, i.e., units compatible with administration using the devices disclosed infra.

A pharmaceutical formulation according to the present disclosure may additionally comprise inert constituents including pharmaceutically-acceptable excipients, carriers, solvents, diluents, fillers, salts, and/or other materials well-known in the art, the selection of which depends upon the dosage form utilized, the condition being treated, the particular purpose to be achieved according to the determination of the ordinarily skilled artisan in the field and the properties of such additives. The stabilized hemoglobin solutions according to the present disclosure may include one or more pharmaceutically acceptable carriers and/or excipients.

Pharmaceutically acceptable excipients include any and all solvents, diluents or other liquid vehicles, dispersion or suspension aids, surface active agents, isotonic agents, thickening or emulsifying agents, preservatives, solid binders, lubricants and the like, as suited to the particular dosage form desired. Examples of excipients include sodium chloride and physiologically-acceptable buffers.

The pharmaceutically acceptable carrier is preferably nontoxic, inert and compatible with hemoglobin. Examples of such carriers include, but are not limited to, water, balanced saline solution, physiologic saline solution (e.g., Lactated Ringer's solution, Hartman's solution, etc.), dextrose solution and the like.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, mannitol, sorbitol, inositol, sodium chloride, etc., and combinations thereof.

In some embodiments, a stabilized hemoglobin solution or a formulation thereof may additionally comprise sodium chloride, potassium chloride, calcium chloride, sodium hydroxide, N-acetyl-L-cysteine, sodium lactate, sodium borate, and/or tris. The stabilized hemoglobin solution, or pharmaceutical formulation thereof, may further comprise a formulation buffer comprising borate or another suitable buffering agent. The borate may be reduced.

In some embodiments, the stabilized hemoglobin solution may comprise one or more electrolytes. Electrolytes useful in the present disclosure include Na, Cl, K, and the like.

In some embodiments, the stabilized hemoglobin is present within the pharmaceutical formulation in an effective amount, e.g., a therapeutically effective amount or a prophylactically effective amount.

Pharmaceutical formulations can be prepared as a single unit dose, and/or as a plurality of single unit doses. As used herein, a “unit dose” is a discrete amount of the pharmaceutical composition comprising a predetermined amount of the stabilized hemoglobin. The amount of the stabilized hemoglobin is generally equal to the dosage of hemoglobin which would be administered to a subject and/or a convenient fraction of such a dosage such as, for example, one-half or one-third of such a dosage.

Relative amounts of the stabilized hemoglobin, the pharmaceutically acceptable carrier, and/or any additional ingredients in a pharmaceutical composition of the invention will vary, depending upon the identity, size, and/or condition of the subject treated and further depending upon the route by which the composition is to be administered. By way of example, the formulation may comprise between 0.1% and 100% (w/w) active ingredient.

The pharmaceutical formulations of the present disclosure may be formulated for administration to a subject by any means. In some embodiments, formulations may be prepared for administration by routes including oral, by aerosol, by transdermal adsorption, by adsorption through a mucus membrane or by injection. In some embodiments, the formulations are prepared for parenteral administration. In some embodiments, the formulations are prepared for intravenous administration. In some embodiments, the formulation is prepared for intraosseous administration.

### Devices for Administering Stabilized Hemoglobin Compositions

Another aspect of the present disclosure provides devices for the administration of the disclosed stabilized hemoglobin solutions and pharmaceutical formulations thereof.

In some embodiments, the device is an injection device. In some embodiments, the device comprises one or more of a needle, an injection pen, an intravenous (IV) line, a central IV line, a syringe, a catheter, and a blood exchanging and/or filtering device.

In some embodiments, the device is intended for administration by an individual who is not a medical professional. In some embodiments, the device is intended for administration outside of a hospital setting. In some embodiments, the device is intended for administration in an emergency situation. In some embodiments, the device is intended for administration in an ambulance.

In some embodiments, the device comprises a preloaded self-injection device. In some embodiments, the device comprises a preloaded self-injection pen.

In some embodiments, the device comprises one or more therapeutically effective doses. In some embodiments, the device comprises one or more unit doses. In some embodiments, the one or more unit doses comprise of volume of between 10 mL and 30 mL, inclusive of the endpoints. In some embodiments, the one or more unit doses comprise a volume of between 18 mL and 25 mL, inclusive of the endpoints.

In some embodiments, the injection device comprises a metering device. In some embodiments, the injection device is operably linked to a metering device. In some embodiments, the injection device may be connected to a metering device.

In some embodiments, the injection device may comprise a titrated dose. Metering and/or titrating the stabilized hemoglobin composition and/or formulation may be performed in conjunction with monitoring one or more physiological symptoms of the subject being treated. The one or more physiological symptoms may be selected from: blood pressure, core temperature, liver tissue oxygenation tension, respiration rate, urine output, stroke volume, heart rate, cardiac output, peak systolic blood flow velocity, arterial PO₂, arterial PCO₂, arterial pH, and arterial base excess.

In some embodiments, the injection device comprises one or more compartments, each capable of maintaining a preloaded volume of the stabilized hemoglobin solution, and each capable of delivering to a subject a distinct volume of the composition, wherein the volume of the composition in each compartment may be delivered simultaneously or sequentially.

In some embodiments, the injection device comprises one or more compartments, each capable of comprising a distinct amount of the formulation buffer to selectively dilute the stabilized hemoglobin solution and/or formulation to a predetermined final concentration for each compartment.

In some embodiments, the injection device provides an escalating or de-escalating dosage regime by injecting from each of the one or more compartments, sequentially, (a) an increasing or a decreasing volume of a stabilized hemoglobin solution and/or formulation, or (b) an increasing or a decreasing concentration of a stabilized hemoglobin solution and/or formulation, respectively.

### Uses of Stabilized Hemoglobin Compositions & Devices

Another aspect of the present disclosure is related to the use of the disclosed solutions, pharmaceutical formulations, and devices in the treatment of a subject in need thereof.

### Subjects

The present solutions, pharmaceutical formulations, and devices may be used to treat a variety of different subjects. In some embodiments, the subject is a human. In some embodiments, the subject is a non-human animal. In some embodiments, the non-human animal is a non-human vertebrate, a non-human primate, a cetacean, a mammal, a reptile, a bird, an amphibian, or a fish. In some embodiments, the non-human animal is a canine or a feline. In some embodiments, the non-human animal is a bovine. In some embodiments, the non-human animal is a mustelid, a captive mustelid, a rodent, a captive rodent, a raptor, or a captive bird. In some embodiments, the captive bird is of the order Psittaciformes, Passeriformes or Columbiformes.

### Diseases and Conditions

The present solutions, formulations, and devices may be used in the treatment of a variety of conditions or diseases, including anemia, hypoxia, and hemoglobinopathies. Hemoglobinopathies encompass a number of anemias of genetic origin in which there is a decreased production and/or increased destruction (hemolysis) of red blood cells (RBCs). These also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickled) hemoglobin (HbS). HbS RBCs are more fragile than normal RBCs and undergo hemolysis more readily, leading eventually to anemia (Atweh, Semin. Hematol. 38(4):367-73 (2001)).

In some embodiments, the present solutions, formulations, and devices may be used to treat anemia. In some embodiments, the anemia may be aplastic anemia, iron deficiency anemia, sickle cell anemia, thalassemia, or vitamin deficiency anemia. In some embodiments, the anemia is characterized by fatigue, weakness, pale or yellowish skin, irregular heartbeats, shortness of breath, dizziness or lightheadedness, chest pain, cold hands and feet, headaches. Anemia may result from the loss of red blood cells, inadequate red blood cell generation, or excessive red blood cell lysis.

In some embodiments, the present solutions, formulations, and devices may be used to treat iron deficiency anemia. Iron deficiency anemia may be caused by a shortage of iron which is required to make hemoglobin. This type of anemia is common among many pregnant women. It is also caused by blood loss, such as from heavy menstrual bleeding, an ulcer, cancer and regular use of some over-the-counter pain relievers, especially aspirin, which can cause inflammation of the stomach lining resulting in blood loss.

In some embodiments, the present solutions, formulations, and devices may be used to treat vitamin deficiency anemia, also known as pernicious anemia. Vitamin deficiency anemia may result from a lack of sufficient folate, vitamin B-12, or other important vitamins. The deficiency may result from an inadequate dietary supply or from an inability to absorb the required vitamins.

In some embodiments, the present solutions, formulations, and devices may be used to treat anemia of inflammation. Certain diseases—including cancer, HIV/AIDS, rheumatoid arthritis, kidney disease, Crohn's disease and other acute or chronic inflammatory diseases—can interfere with the production of red blood cells.

In some embodiments, the present solutions, formulations, and devices may be used to treat aplastic anemia. This rare, life-threatening anemia occurs when the body doesn't produce enough red blood cells. Causes of aplastic anemia include infections, certain medicines, autoimmune diseases and exposure to toxic chemicals.

In some embodiments, the present solutions, formulations, and devices may be used to treat anemias associated with bone marrow disease. A variety of diseases, such as leukemia and myelofibrosis, can cause anemia by affecting blood production in your bone marrow. The effects of these types of cancer and cancer-like disorders vary from mild to life-threatening. In some embodiments, the present compositions may be used to treat a subject who has experienced blood loss as a result of blood cancer or treatment for cancer.

In some embodiments, the present solutions, formulations, and devices may be used to treat hemolytic anemias. This group of anemias develops when red blood cells are destroyed faster than bone marrow can replace them. Certain blood diseases increase red blood cell destruction. Hemolytic anemia can be inherited or developed later in life.

In some embodiments, the present solutions, formulations, and devices may be used to treat sickle cell anemia. This inherited and sometimes serious condition is a hemolytic anemia. It's caused by a defective form of hemoglobin that forces red blood cells to assume an abnormal crescent (sickle) shape. These irregular blood cells die prematurely, resulting in a chronic shortage of red blood cells.

In some embodiments, the present solutions, formulations, and devices may be used in the treatment of hypoxia. Hypoxia is a condition in which the body or a region of the body is deprived of adequate oxygen supply at the tissue level. Hypoxia may be classified as either generalized, affecting the whole body, or local, affecting a region of the body. In some embodiments, the present treatments may be used to treat generalized hypoxia. In some embodiments, the present treatments may be used to treat local hypoxia. Although hypoxia may be a pathological condition, variations in arterial oxygen concentrations can be part of the normal physiology, for example, during hypoventilation training or strenuous physical exercise. In some embodiments, the present treatments are used to treat an individual experiencing hypoxia as a result of strenuous physical activity.

In some embodiments, the present treatments are used to treat an individual who has ascended to high altitude. Generalized hypoxia occurs in healthy people when they ascend to high altitude, where it causes altitude sickness leading to potentially fatal complications: high altitude pulmonary edema (HAPE) and high altitude cerebral edema (HACE). In some embodiments, the present treatments are used to treat a subject experiencing altitude sickness, HAPE, or HACE.

In some embodiments, the present treatments are used to treat an individual who has dived underwater. Hypoxia may occur in healthy individuals when breathing mixtures of gases with a low oxygen content, e.g. while diving underwater especially when using closed-circuit rebreather systems that control the amount of oxygen in the supplied air.

In some embodiments, the present treatment may ameliorate or lessen one or more symptoms of hypoxia. In hypoxia, gradual onset symptoms include fatigue, numbness/tingling of extremities, nausea, and cerebral anoxia. In severe hypoxia, or hypoxia of very rapid onset, ataxia, confusion/disorientation/hallucinations/behavioral change, severe headaches/reduced level of consciousness, papilloedema, breathlessness, pallor, tachycardia, and pulmonary hypertension eventually leading to the late signs cyanosis, slow heart rate/corpulmonale, and low blood pressure followed by heart failure eventually leading to shock and death. In some embodiments, the present treatments may ameliorate or prevent one or more side effects of hypoxia.

In some embodiments, the subject has experienced blood loss from an injury, blood loss from a medical intervention, hemolysis or reduced hematopoiesis. In some embodiments, the present treatments return arterial flow, volume, and/or pressure to acceptable levels, and oxygenation of tissues to acceptable levels. In some embodiments, the compositions may be used to treat a subject suffering exsanguination, internal injury (e.g., internal organ injury), hemorrhage, hemorrhagic shock, or traumatic brain injury. In some embodiments, the present compositions may be used as a means of resuscitation.

In some embodiments, the stabilized hemoglobin solutions may be administered in the absence of whole blood transfusion. In some embodiments, the stabilized hemoglobin solution may be administered in conjunction with whole blood transfusion. In some embodiments, the stabilized hemoglobin solution may be administered prior to or following whole blood transfusion.

In some embodiments, the present compositions may be used for preoperative hemodilution. In some embodiments, the present compositions may be used to treat a subject prior to an operation. In some embodiments, the operation is, e.g., aortic surgery, liver resection, or organ transplant.

### Administration Routes

The stabilized hemoglobin solutions, pharmaceutical formulations thereof may be administered by any route. In some embodiments, the administration is oral, topical, parenteral, enteral, transdermal, intradermal, intraocular, intravitreal, subcutaneous, intravenous, or intraosseous. In some embodiments, the administration is parenteral. In some embodiments, the administration is intravenous. In some embodiments, the administration is intraosseous.

### Dosage and Dosing Schedules

Administration may comprise administering a therapeutically effective amount in one or more doses. In some embodiments, the stabilized hemoglobin solution or pharmaceutical formulation may be administered in a single dose or in two or more doses. In some embodiments, the stabilized hemoglobin solution is metered into a subject in need thereof. In some embodiments, administration comprises titrating stabilized hemoglobin composition instead of a single bolus injection. Metering and/or titrating the stabilized hemoglobin composition and/or formulation, e.g. with an injection device of the present disclosure, may be performed in conjunction with monitoring one or more physiological symptoms of the subject being treated. The one or more physiological symptoms may be selected from: blood pressure, core temperature, liver tissue oxygenation tension, respiration rate, urine output, stroke volume, heart rate, cardiac output, peak systolic blood flow velocity, arterial PO₂, arterial PCO₂, arterial pH, and arterial base excess.

In some embodiments, the present stabilized hemoglobin compositions may be administered in a repeat dosing schedule to achieve a plasma level of 0.3-0.4 g/dL. Additional doses may be administered to maintain this concentration over time. In some embodiments, the present stabilized hemoglobin solutions may be formulated in a concentration range of 15-20 g/dL and administered to a subject in a total dosage sufficient to achieve 0.3-0.4 g/dL plasma concentration of stabilized hemoglobin. The total dosage may be administered in a single dose, in several doses, or according to a repeat dosing schedule. In a repeat dosing schedule, doses may be administered repeatedly, separated by a period of seconds, minutes, hours, or days. In some embodiments, repeat doses are separated by a period of about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 1.5 days, about 2 days, about 3 days, about 4 days, about 5 days, about 6 days, or about one week. In some embodiments, subsequent doses are separated by a period of about 6 hours. In some embodiments, subsequent doses are separated by a period of about 12 hours. In some embodiments, subsequent doses are separated by a period of about 1 day. In some embodiments, plasma hemoglobin level is monitored to determine timing of a subsequent dose. In some embodiments, stabilized hemoglobin level is monitored to determine timing of a subsequent dose in order to achieve and maintain a concentration of 0.3-0.4 g/dL of stabilized hemoglobin solution.

### In Vivo Characteristics

The stabilized hemoglobin compositions disclosed herein may be used as an oxygen carrier and/or blood substitute. In some embodiments, the substance is substantially free of endotoxins, has the property of reversibly binding gaseous ligands such as oxygen and is useful for transporting and supplying oxygen to vital tissues and organs. As such, in some embodiments, the stabilized hemoglobin composition of the present invention is useful as a blood expander and resuscitating fluid in the management of disease and for maintaining circulatory integrity where needed, i.e., in response to sudden and massive blood loss.

In some embodiments, the stabilized hemoglobin composition is substantially endotoxin free and pyrogen free. In some embodiments, it does not cause any of the following abnormal and detrimental chemical and physiologic functions in vivo: (1) does not activate complement; (2) does not cause hemorrhagic disorders; (3) does not cause abnormal platelet function or aggregation; (4) does not cause abnormal prothrombin times (PT); (5) does not cause abnormal partial thromboplastin times; (6) does not interfere with blood typing or cross-matching; (7) is non-toxic to the kidneys in 3.5 grams per kilogram per body weight or 8 grams per deciliter circulating blood volume; (8) exhibits circulating persistence of at least seven days; and (9) acts as a stimulus to accelerated erythropoiesis.

### EXAMPLES

### Example 1: Production of Highly Concentrated, Deoxygenated, Stabilized Hemoglobin Compositions

### Blood Unloading:

The sourced blood material (defibrinated or citrated) is diluted with a suitable, physiologically compatible buffer solution through a static mixer. The blood is pumped through a 50 μm blood strainer and a 60 μm depth filter to remove extraneous materials or large aggregates if needed.

The Ultrafilter skid is flushed with buffer prior to use. The filtered blood is further diluted then concentrated to the original loading volume then washed with 7 volumes of buffer solution using the Ultrafilter Skid.

The washed red cell solution is pumped into the centrifuge and spun at sufficient rotational speed and duration so as to separate the heavy phase containing the red blood cells (RBC) from the lighter phase containing the remaining plasma, and buffer. The heavy phase is discharged into a product collection receptacle. The discharge of the cell containing phase from the centrifuge may cause the disruption and lysing of the red blood cells, for example, by impact upon discharge from the centrifuge into the product collection vessel. Alternatively, the discharge of the red blood cell may necessitate the lysing of the cells by another method. The cell solution is pumped from the product collection receptacle to a red blood cell receptacle. If lysing of the red blood cells is required, the red blood cells may be diluted inline with Depyrogenated Water (DPW) through a static mixer while being transferred to the RBC receptacle. In this embodiment, the red blood cells may be lysed due to the rapid change in osmotic pressure, as the depyrogenated water is added during transfer.

### Filtration and Storage:

The collected cells are sampled, tested for hemoglobin, then adjusted to 14.0-18.0 g/dL using DPW.

The 100 KDA and 30 kDA skids are flushed with DPW prior to use. The red blood cell solution is diafiltered using a 100 kDa membrane and about 11 volumes of DPW. This operation eliminates cellular debris larger than 100 kDa. The permeated hemoglobin-containing solution is simultaneously ultrafiltered using a 30 kDa membrane to concentrate the hemoglobin and to remove smaller debris and micro-contaminants. The hemoglobin is analyzed and ultrafiltration is continued until the intermediate is concentrated to approximately 13 g/dl. The hemoglobin, at 64 kDa, is retained after these two steps. The concentrated hemoglobin is sampled for in-process testing.

After testing, the hemoglobin is pumped through a 0.5 μm and a 0.22 μm clarification filter into a receptacle. The receptacle contents are sampled then the receptacle is relocated to a 2-8° C. cold room.

### Chromatography:

The crude hemoglobin is removed from refrigerated storage for chromatographic purification.

The column is equilibrated with Buffer A (2.42 g/L Tris, pH 9) prior to purification. The product is fed onto the column, with a bed height of 30 cm with a linear flow rate of 400 cm/hr. The column is then washed with buffer A followed by a pH gradient elution with buffer A transitioning to buffer B (6.05 g/L Tris, pH 7). This buffer elutes loosely bound non-hemoglobin components which are sent to the waste stream. The product fraction is collected by recognizing a change to OD or absorbance in a suitable wavelength for detecting hemoglobin, typically 541 nm and, more notably, 577 nm, are usefully monitored for indicating the presence of hemoglobin.

### Deoxygenation/Concentration:

The concentrated solution is transferred to a degassing vessel and the ionic strength is adjusted to >200 mM using Buffer C (2.42 g/L Tris, 58.38 g/L NaCl pH 8.9). The solution is then deoxygenated by diafiltration against a degassing membrane with nitrogen flowing across the opposite side of the membrane.

The deoxygenated solution is diafiltered into deoxygenated storage buffer (Phosphate solution with 2 g/L N-acetyl-L-cysteine) using a 30 kDa MWCO membrane filter and 3 diavolumes of the deoxygenated storage buffer.

The deoxygenated hemoglobin intermediate is sampled for in-process testing and filtered into a storage bag using 0.5 μm and 0.22 μm filters. This intermediate is stable for up to 60 days at 17-22° C.

### Compound Load, Mixing, and Polymerization:

This process begins by charging deoxygenated water for injection (WFI) (˜½ intermediate volume), USP into a reactor vessel with mixing/recirculation and warmed to 42° C. The hemoglobin intermediate is added to the reactor vessel, chased by 2.5 volumes of additional deoxygenated WFI, USP.

Once temperature is achieved, the hemoglobin intermediate is transferred to another tank. 0.62% Glutaraldehyde Activation Solution is added to the hemoglobin solution as it is transferred to the other tank in order to polymerize the hemoglobin. Once the polymerization time is complete, the polymerized hemoglobin solution is cooled to 20° C.

### Diafiltration, Concentration, and Compound Storage:

The polymerized hemoglobin solution is concentrated to ˜8 g/dL and diafiltered using a 30 kDa MWCO membrane with 3 diavolumes of Borate buffer (4.58 g/L sodium borate 10-hydrate, 0.91 g/L sodium hydroxide, pH 10.4-10.6) to adjust the pH of the solution. The polymerized hemoglobin is then recirculated across a deoxygenation filter against a cross-flow of nitrogen to remove hydrogen from the process.

The recirculating polymerized hemoglobin solution is then quenched by the addition of Quench Solution (9.00-9.95 g sodium borohydride/kg borate buffer) and slowed to recirculate through a 30 kDa MWCO filter and a deoxygenation filter for 1 hour. This step concentrates the hemoglobin to approximately 70-200 g/L, and may be desirably concentrated to suitable ranges including: 70-100 g/L, 85-125 g/L, 95-150 g/L, or 150-200 g/L.

The solution is buffer exchanged by diafiltration with 6 diavolumes of Diafiltration Buffer A (6.67 g/L sodium chloride, 0.30 g/L potassium chloride, 0.20 g/L calcium chloride dihydrate, 0.445 g/L sodium hydroxide. 2.02 g/L N-acetyl-L-cysteine. 3.07 g/L sodium lactate) with the continued use of a deoxygenation filter.

Finally, the material is buffer exchanged with 3 diavolumes of Diafiltration Buffer C (6.73 g/L sodium chloride, 0.30 g/L potassium chloride, 0.20 g/L calcium chloride dehydrate 0.512 g/L sodium hydroxide. 2.03 g/L N-acety 1-L-cysteine. 3.08 g/L sodium lactate, pH 7.75±0.15).

The resulting batch of Stabilized Hemoglobin Drug Substance is filtered into deoxygenated Drug Substance containers using pre-wetted (deoxygenated WFI) 0.22 μm filters and transferred to storage. The bulk DS is stored at 15-30° C. until further transport or use.

### Example 2: In Vitro Characterization of Stabilized Hemoglobin Composition

Stabilized hemoglobin composition was formulated as described in Example 1 and then evaluated for potency, purity, and identity. An illustrative evaluation of an exemplary batch of stabilized hemoglobin composition is provided in Table 1 below. As demonstrated in Table 1, the stabilized hemoglobin composition had test results within specified parameters for all tested metrics.

TABLE 1
a.
Potency, Purity, and Identity Test Results

| | Approved Test | | | |
|---|---|---|---|---|
| Release Tests | Methods | Unit | Specification | Test Result |
| 1. Potency | | | | |
| Total Hb | Co-oximetry | g/dL | 5.5-7.5 | 6.4 |
| Met Hb | Co-oximetry | % | <10 | 0.4 |
| Oxy Hb | Co-oximetry | % | <10 | 1.8 |
| 2. Purity | | | | |
| Sterility | Sterility test | N/A | Pass | Pass |
| Endotoxin Level | Kinetic turbidimetric | EU/mL | <0.05 | <0.05 |
| Molecular Weight | | | | |
| Distribution | | | | |
| MW <32,000 | HPLC-SEC | % | <5 | 4.44 |
| 3. Identity | | | | |
| Appearance | Visual | N/A | Deep Purple | Deep Purple |
| pH | Potentiometry | N/A | 7.6-7.9 | 7.69 |
| | | | @18-22° C. | |
| Ion Concentration | | | | |
| Na⁺ | Ion selective electrode | mM | 145-160 | 154 |
| K⁺ | Ion selective electrode | mM | 3.5-5.5 | 3.9 |
| Ca²⁻ | Ion selective electrode | mM | 0.5-1.5 | 0.77 |

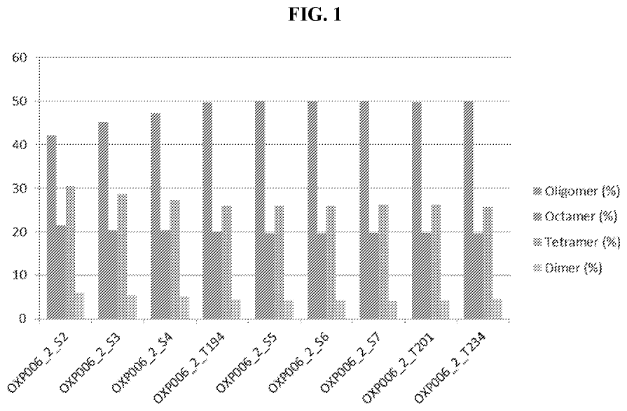

An analysis of oligomer/octamer/tetramer/dimer distribution was also performed on batches that had been cross-linked with glutaraldehyde and reduced with NaBH₄, demonstrating acceptable levels of each species of hemoglobin with different levels of crosslinking agents and reducing agents. Results are shown in Table 2 and FIG. 1.

TABLE 2
b.
Component Percentage Comparison

| Sample Name | Oligomer (%) | Octamer (%) | Tetramer (%) | Dimer (%) |
|---|---|---|---|---|
| OXP006_2_S2 | 42.11 | 21.46 | 30.42 | 6.01 |
| OXP006_2_S3 | 45.33 | 20.40 | 28.72 | 5.56 |
| OXP006_2_S4 | 47.23 | 20.38 | 27.25 | 5.14 |
| OXP006_2_T194 | 49.62 | 19.92 | 26.06 | 4.40 |
| OXP006_2_S5 | 50.12 | 19.62 | 26.04 | 4.22 |
| OXP006_2_S6 | 50.07 | 19.66 | 26.01 | 4.26 |
| OXP006_2_S7 | 50.05 | 19.73 | 26.15 | 4.07 |
| OXP006_2_T201 | 49.86 | 19.75 | 26.10 | 4.29 |
| OXP006_2_T234 | 50.18 | 19.59 | 25.69 | 4.53 |

### Example 3: In Vitro Characterization of Highly Concentrated Stabilized Hemoglobin Solution for Human Use

A highly concentrated stabilized hemoglobin solution, at a concentration between 150 g/L and 200 g/L, is tested as in Example 2 for Potency, Purity, Identity, and Component Distribution. Results reveal acceptable values for each tested parameter.

### Example 4: Pharmacokinetics of the Stabilized Hemoglobin Composition in Anesthetized Animals

### Animals:

Male Wistar rats (300-400 g body weight) are used in all experiments. Animals are purchased from Charles River (Margate, Kent UK) and certified healthy and pathogen-free on arrival at University College London. One week prior to experimentation, animals are housed in standard cages of four individuals on a twelve-hour light/dark cycle, with food and water ad libitum.

### Surgical Procedures:

All animals are anaesthetized by isoflurane in room air (Abbott, Maidenhead, Berks, UK); 5% for induction, 2% for surgical procedures and 1.5% for maintenance. They are placed on a heated mat (Harvard Apparatus, Cambridge, Cambs, UK) to maintain rectal temperature at 37° C. For the anesthetized model, the left common carotid artery and right internal jugular vein are cannulated using 0.96 mm outside diameter PVC tubing catheter (Scientific Commodities Inc., Lake Havasu City, AZ, USA). The arterial line is connected to a pressure transducer (Powerlab; AD Instruments, Chalgrove, Oxon, UK) for continuous monitoring of mean arterial blood pressure, and the venous line is used for administration of Ringer's lactate±the stabilized hemoglobin composition. The trachea is cannulated to suction and secure the airway in these spontaneously breathing animals. The bladder is cannulated through a keyhole laparotomy to measure urine output and renal excretion of the stabilized hemoglobin composition. The tissue partial pressure of oxygen (tPO₂) is measured in liver using fiber-optic probes and an Oxylite™ system (Oxford Optronix, Oxon, UK). This tissue bed is chosen as it is the most sensitive to hemodynamic perturbations and cardiorespiratory challenges. Liver tPO₂ is monitored by placing the sensor (through a keyhole laparotomy) in an airtight pocket between two lobes. In these nonrecovery experiments, euthanasia at experiment-end is performed using IV sodium pentobarbitone.

### Anesthetized Dose-Finding and Pharmacokinetic Study:

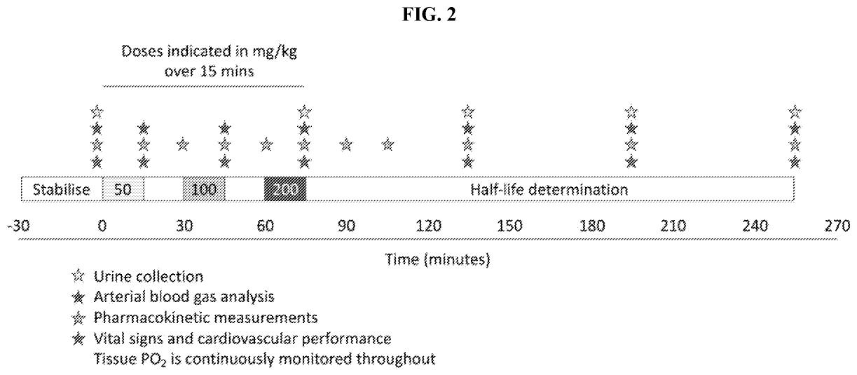

Following surgery and 30 min stabilization period, animals receive increasing IV infusions of the stabilized hemoglobin composition (n=6) or an equivalent volume of Ringer's lactate (2.5 ml/kg; n=6), administered over 15 minutes (FIG. 2). the stabilized hemoglobin composition is supplemented with Ringer's lactate (through a Y-shaped connector) such that the total infusion rate is 10 ml/kg/h. Infusions of 50, 100 and 200 mg/kg of the stabilized hemoglobin composition are examined with doses escalated every 30 minutes, using a Harvard Apparatus Fluid pump, 21-gauge needles and (5 ml) Terumo syringes. After the highest infusion, animals are maintained for three hours.

At baseline (i.e., pre-infusion), a series of measurements are made, as detailed in Table 3 and FIG. 2. Collection of urine and blood sampling for baseline pharmacokinetic measurements is additionally performed. Whole blood (0.3 ml) is removed into ethylenediamine tetraacetic acid (EDTA)-containing syringes (final concentration; 2.5 mM) and centrifuged (1300×g). The plasma fraction is then either frozen in liquid nitrogen for subsequent analysis spectrophotometrically or, when available, processed immediately using a Hemocue device.

Blood for pharmacokinetics is taken at the beginning and end of each of the stabilized hemoglobin composition infusion and at frequent intervals up to three hours after the highest infusion. Arterial blood gas analysis, vital signs and cardiovascular performance measurements are taken at baseline, the end of each infusion, then hourly. Urine is collected at baseline, at the end of the highest infusion, then hourly. To determine fractional renal excretion, a mass-balance approach will be utilized. This is accomplished by calculation of the number of moles of the stabilized hemoglobin composition excreted in urine, expressed as a fraction against the number of moles injected.

TABLE 3
Measurements of cardiorespiratory status, tissue oxygen tension and pharmacokinetics in
plasma and urine in anesthetized animal study.

| | Vital | | | | | | | | |
|---|---|---|---|---|---|---|---|---|---|
| | signs | | Arterial blood gas analysis | | | | | | |
| | Blood | Cardiac | | Substrates/ | | | | | |
| | pressure | performance | | perfusion | | | | | |
| | Heart | Stroke | Oxygenation | markers | Electrolytes | | | | |
| | rate | volume | PaO₂ | pH | K⁺ | Miscellaneous | | | |
| | Core | Flow | PaCO₂ | Base | Na⁺ | Liver | Plasma | | Urine |
| Time | temperature | velocity | Haemoglobin | excess | Ca²⁺ | tissue | (OxyPly-V) | | (OxyPly-V) |
| (mins) | Respiration | Cardiac | Oxy-Hb | Lactate | Cl⁻ | oxygen | or | Urine | or |
| T | rate | output | Met-Hb | Glucose | HCO₃⁻ | tension | (Oxyglobin) | output | (Oxyglobin) |
| 0 | x | x | x | x | x | x | x | x | x |
| 15 | x | x | x | x | x | x | x | | |
| 30 | | | | | | x | x | | |
| 45 | x | x | x | x | x | x | x | | |
| 60 | | | | | | x | x | | |
| 75 | x | x | x | x | x | x | x | x | x |
| 90 | | | | | | x | x | | |
| 105 | | | | | | x | x | | |
| 120 | | | | | | | | | |
| 135 | x | x | x | x | x | x | x | x | x |
| 150 | | | | | | | | | |
| 165 | | | | | | | | | |
| 180 | | | | | | | | | |
| 195 | x | x | x | x | x | x | x | x | x |
| 210 | | | | | | | | | |
| 225 | | | | | | | | | |
| 240 | | | | | | | | | |
| 255 | x | x | x | x | x | x | x | x | x |

In Table 3, the following abbreviations are employed: Ca2+, calcium; Cl−, chloride; Hb, hemoglobin; HCO3−, bicarbonate; K+, potassium; PaO₂ and PaCO₂, arterial partial pressures of carbon dioxide and oxygen, respectively. Oxy- and met-Hb denote oxyhemoglobin and methemoglobin, respectively. Note that ‘flow velocity’ is the peak systolic blood flow velocity, a cardiac contractility marker. Tissue oxygen tension is presented at the times corresponding to plasma drug levels. T=time in minutes.

### Results:

A study was performed according to the foregoing description. Numerous parameters, including levels of hemoglobin, arterial PO₂, arterial PCO₂, and oxyhemoglobin, were maintained within appropriate limits throughout the experiment.

### Example 5: Pharmacokinetic Study of the Stabilized Hemoglobin Composition in Awake Animals

### Surgical Procedures:

For the recovery model, animals are anaesthetized as detailed in Example 4. Vascular lines (left common carotid artery and right internal jugular vein) are cannulated and tunneled subcutaneously to the nape of the neck. These are attached to a swivel tether system that allows the animal, on recovery from anesthesia, unimpeded movement around its cage. Buprenorphine (0.05 mg/kg s.c.) is used for analgesia, administered at the beginning of surgery.

### Pharmacokinetic Study in Awake Animals:

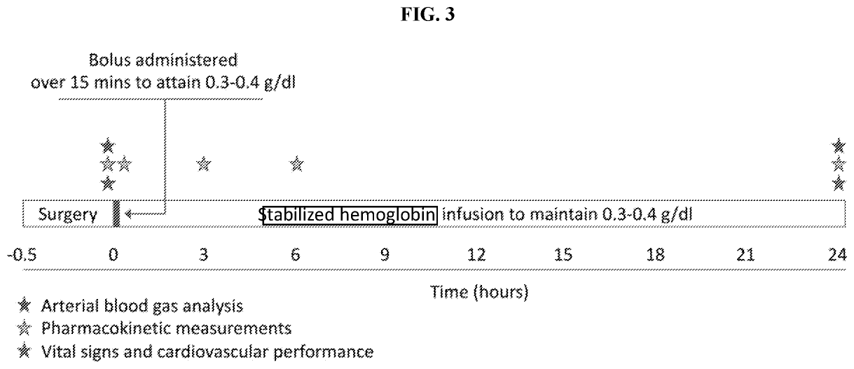

Vital signs, cardiac performance and arterial blood gas analyses are assessed following a 30 min stabilization period post-surgery. Additional blood is removed for pharmacokinetic analyses from the arterial line. On recovery from anesthesia, animals are administered a bolus of the stabilized hemoglobin composition (n=6) or an equivalent volume of Ringer's lactate solution (n=6) over 15 mins to attain plasma levels of 0.3-0.4 g/dl of the stabilized hemoglobin composition. The dose-level required is determined based on the outcome of Example 4. A further blood sample is taken at 15 mins to confirm plasma concentrations. This is followed by a continuous infusion over 24 h to maintain plasma levels at 0.3-0.4 g/dl, with blood sampling at 3, 6 and 24 h. At experiment end (24 h), animals are re-anesthetized; vital signs and cardiac performance are reassessed, and blood removed for arterial blood gas and pharmacokinetic measurements. The planned measurements are also shown in Table 4 and FIG. 3.

### Pharmacokinetics:

If a hemocue device is not available, absorbance (λ 577 and 635) of plasma and urine samples will be assessed using a microplate reader and BioTek (Gen5) software (Synergy 2, North Star Scientific, Sandy, Beds, UK). Concentrations (CMAX, TMAX) of the stabilized hemoglobin composition are measured following derivation against standard curves. The typical composition. The dose-level required is determined based on the outcome of Example 4. A further blood sample is taken at 15 minutes to confirm plasma concentrations. This is followed by a continuous infusion over 24 h to maintain plasma levels at 0.3-0.4 g/dl, with blood sampling at 3, 6 and 24 h. At experiment end (24 h), animals are re-anesthetized; vital signs and cardiac performance are reassessed, and blood removed for arterial blood gas and pharmacokinetic measurements. The planned measurements are also shown in Table 4 and FIG. 3.

### Pharmacokinetics:

If a hemocue device is not available, absorbance (λ 577 and 635) of plasma and urine samples will be assessed using a microplate reader and BioTek (Gen5) software (Synergy 2, North Star Scientific, Sandy, Beds, UK). Concentrations (CMAX, TMAX) of the stabilized hemoglobin composition are measured following derivation against standard curves. The typical oxyhemoglobin absorbance spectrum shows distinct peaks at 541 nm and, more notably, 577 nm. Upon oxidation, oxyhemoglobin (Fe2+) is converted to methemoglobin (Fe3+); which is less absorbent at 577 nm and has an additional absorbance peak at 635 nm. If any absorbance is noted at 635 nm, indicative of the presence of methemoglobin (either in the stabilized hemoglobin composition or plasma/urine samples), Drabkin's method is employed to ensure total hemoglobin is measured.

Drabkin's method (if required) is performed as follows:
(1) Conversion of all hemoglobin to methemoglobin with the addition of K-ferricyanide (0.6 mM).
(2) Addition of potassium cyanide (0.77 mM) to convert all methemoglobin to cyanomethemoglobin.
(3) Assay cyanomethemoglobin spectrophotometrically at 540 nm.

### Data and Statistics:

Animals are randomly (drawing of lots) assigned to receive the stabilized hemoglobin composition (n=6) or to act as controls (Ringer's lactate; n=6). Data is presented as mean±standard error or median, quartiles, and range. Anticipated statistical analysis comprises a twoway repeated measures ANOVA followed by Bonferroni's post-hoc test. Pharmacokinetic data (determination of half-life and exposure; AUC) is calculated using a one- or two-phase decay curve and least squares fitting method. All statistical analyses are two-tailed and performed using Prism 7.0.1 software (GraphPad Software, San Diego, CA). Multiplicity adjusted p-values <0.05 are considered statistically significant.

The exemplary in vivo studies described in Examples 4 and 5 are also performed with highly concentrated and stabilized hemoglobin composition at a concentration of 150 g/L-200 g/L.

TABLE 4
Measurements of cardiorespiratory status and plasma drug levels in Study 2.

| | Vital | | | | | |
|---|---|---|---|---|---|---|
| | signs | | Arterial blood gas analysis | | | |
| | Blood | Cardiac | | Substrates/ | | |
| | pressure | performance | | perfusion | | |
| | Heart | Stroke | Oxygenation | markers | Electrolytes | |
| | rate | volume | PaO₂ | pH | K⁺ | |
| | Core | Flow | PaCO₂ | Base | Na⁺ | Misc. |
| Time | temperature | velocity | Haemoglobin | excess | Ca²⁺ | (OxyPly-V) |
| (mins) | Respiration | Cardiac | Oxy-Hb | Lactate | Cl⁻ | or |
| T | rate | output | Met-Hb | Glucose | HCO₃⁻ | (Oxyglobin) |
| 0 | x | x | x | x | x | x |
| 0.25 | | | | | | x |
| 3 | | | | | | x |
| 6 | | | | | | x |
| 24 | x | x | x | x | x | x |

Abbreviations as per Table 3. T=time in hours.

The foregoing illustrates some of the possibilities for practicing the invention. Many other embodiments and fields of use for the depth indicator are possible and within the scope and spirit of the invention. It is, therefore, intended that the foregoing description be regarded as illustrative rather than limiting.

What is claimed is:

1. A composition comprising: a stabilized hemoglobin at a concentration of between 150 grams (g) per Liter (L) (g/L) and 200 g/L, inclusive of the endpoints, wherein the composition comprises less than 0.02 milligrams per milliliter (mg/mL) of dissolved oxygen.

2. A composition comprising a stabilized hemoglobin, wherein the stabilized hemoglobin comprises:
20-35% of the total hemoglobin being in tetrameric form;
15-20% of the total hemoglobin being in octameric form;
40-55% of the total hemoglobin being in greater-than-octameric form;
less than 5% of the total hemoglobin being in dimer form; or
any combination thereof.

3. The composition of claim 2, wherein the composition comprises less than 0.02 mg/mL of dissolved oxygen.

4. The composition of claim 2 or 3, where the stabilized hemoglobin is at a concentration of between 70 and 200 grams per Liter (g/L) inclusive of the endpoints.

5. The composition of claim 4, where the stabilized hemoglobin is at a concentration of between 150 and 200 g/L inclusive of the endpoints.

6. The composition of any one of claims 1-5, wherein the stabilized hemoglobin is stabilized by contacting at least one stabilizing agent selected from a group consisting of: glutaraldehyde, succindialdehyde, activated forms of polyoxyethylene and dextran, α-hydroxy aldehydes, glycolaldehyde, N-maleimido-6-aminocaproyl-(2′-nitro, 4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl) cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl) butyrate, sulfosuccinimidyl 4-(p-maleimidophenyl)butyrate, 1-ethyl-3-(3-dimethylaminopropyl)carbodiimide hydrochloride, N,N′-phenylene dimaleimide, a bis-imidate compound, an acyl diazide compound, an aryl dihalide compound, and combinations thereof.

7. The composition of any one of claims 1-6, wherein the composition is stable at a temperature selected from the group consisting of ambient, refrigerated, above a temperature of at least 4° C., or below a temperature of 30° C.

8. The composition of any one of claims 1-7, wherein greater than about 80% of the stabilized hemoglobin has a molecular weight distribution of between 68 kilodaltons and 500 kilodaltons.

9. The composition of any one of claims 1-8, wherein the composition further comprises a formulation buffer comprising one or more of borate, anti-oxidants, and electrolytes.

10. The composition of claim 9, wherein the borate is reduced.

11. The composition of claim 9, wherein the anti-oxidants comprise N-acetyl-L-cysteine.

12. The composition of claim 9, wherein the electrolytes comprise Na, Cl, and/or K.

13. The composition of any one of claims 1-12, wherein the composition comprises fewer than 0.05 endotoxin units (EU) per milliliter (mL) (EU/mL).

14. The composition of any one of claims 1-13, wherein the hemoglobin comprises hemoglobin isolated or derived from a human, a human cell or a human cell line.

15. The composition of claim 14, wherein the hemoglobin is isolated or derived from no more than 100 variable sources.

16. The composition of claim 14 or 15, wherein the hemoglobin is isolated or derived from harvested red blood cells within 15 days after harvest.

17. The composition of any one of claims 1-13, wherein the hemoglobin comprises hemoglobin isolated or derived from a non-human animal, a non-human cell or a non-human cell line.

18. The composition of claim 17, wherein the hemoglobin is isolated or derived from harvested red blood cells within 10 days after harvest.

19. The composition of claim 17, wherein the non-human animal is a non-human vertebrate, a non-human primate, a cetacean, a mammal, a reptile, a bird, an amphibian, or a fish.

20. The composition of claim 19, wherein the non-human animal is a bovine species.

21. The composition of claim 19, wherein the non-human animal is an ovine species.

22. The composition of claim 19, wherein the non-human animal is a mustelid, a captive mustelid, a rodent, a captive rodent, a raptor, or a captive bird.

23. The composition of claim 22, wherein the captive bird is of the order Psittaciformes, Passeriformes or Columbiformes.

24. The composition of any one of claims 19-23, wherein the non-human animal is not a squab that is raised for food.

25. The composition of any one of claims 1-24, wherein the hemoglobin comprises:
(a) a subunit alpha (α), wherein the subunit α comprises the amino acid sequence of
```
(SEQ ID NO: 1)
    1 MVLSPADKTN VKAAWGKVGA HAGEYGAEAL
        ERMELSFPTT KTYFPHEDLS HGSAQVKGHG
  61 KKVADALTNA VAHVDDMPNA LSALSDLHAH
        KLRVDPVNFK LLSHCLLVTL AAHLPAEFTP
121 AVHASLDKFL ASVSTVLTSK YR,
```
or a sequence having at least 90% identity to the sequence of SEQ ID NO: 1,
or wherein the subunit α is encoded by the nucleic acid sequence of
```
(SEQ ID NO: 2)
    1 actcttctgg tccccacaga ctcagagaga
        acccaccatg gtgctgtctc ctgccgacaa
  61 gaccaacgtc aaggccgcct ggggcaaggt
        tggcgcgcac gctggcgagt atggtgcgga
121 ggccctggag aggatgttcc tgtccttccc
        caccaccaag acctacttcc cgcacttcga
181 cctgagccac ggctctgccc aggttaaggg
        ccacggcaag aaggtggccg acgcgctgac
241 caacgccgtg gcgcacgtgg acgacatgcc
        caacgcgctg tccgccctga gcgacctgca
301 cgcgcacaag cttcgggtgg acccggtcaa
        cttcaagctc ctaagccact gcctgctggt
361 gaccctggcc gcccacctcc ccgccgagtt
        cacccctgcg gtgcacgcct ccctggacaa
421 gttcctggct tctgtgagca ccgtgctgac
        ctccaaatac cgttaagctg gagcctcggt
481 agcagttcct cctgccagat gggcctccca
        acgggccctc ctcccctcct tgcaccggcc
541 cttcctggtc tttgaataaa gtctgagtgg
        gcggc,
```
or a sequence having at least 90% identity to the sequence of SEQ ID NO: 2; or
(b) a subunit beta (β), wherein the subunit β comprises the amino acid sequence of
```
(SEQ ID NO: 3)
    1 MVHLTPEEKS AVTALWGKVN VDEVGGEALG
        RLLVVYPWTQ RFFESFGDLS TPDAVMGNPK
  61 VKAHGKKVLG AFSDGLAHLD NLKGTFATLS
        ELHCDKLHVD PENFRLLGNV LVCVLAHHFG
121 KEFTPPVQAA YQKVVAGVAN ALAHKYH,
```
or a sequence having at least 90% identity to the sequence of SEQ ID NO: 3,
or wherein the subunit β is encoded by the nucleic acid sequence of
```
(SEQ ID NO: 4)
    1 acatttgctt ctgacacaac tgtgttcact
        agcaacctca aacagacacc atggtgcatc
  61 tgactcctga ggagaagtct gccgttactg
        ccctgtgggg caaggtgaac gtggatgaag
121 ttggtggtga ggccctgggc aggctgctgg
        tggtctaccc ttggacccag aggttctttg
181 agtcctttgg ggatctgtcc actcctgatg
        ctgttatggg caaccctaag gtgaaggctc
241 atggcaagaa agtgctcggt gcctttagtg
        atggcctggc tcacctggac aacctcaagg
301 gcacctttgc cacactgagt gagctgcact
        gtgacaagct gcacgtggat cctgagaact
361 tcaggctcct gggcaacgtg ctggtctgtg
        tgctggccca tcactttggc aaagaattca
421 ccccaccagt gcaggctgcc tatcagaaag
        tggtggctgg tgtggctaat gccctggccc
481 acaagtatca ctaagctcgc tttcttgctg
        tccaatttct attaaaggtt cctttgttcc
541 ctaagtccaa ctactaaact gggggatatt
        atgaagggcc ttgagcatct ggattctgcc
601 taataaaaaa catttatttt cattgcaa,
```
or a sequence having at least 90% identity to the sequence of SEQ ID NO: 4;
(c) a subunit gamma (γ), wherein the subunit γ comprises the amino acid sequence of
```
(SEQ ID NO: 5)
    1 MGHFTEEDKA TITSLWGKVN VEDAGGETLG
        RLLVVYPWTQ RFFDSFGNLS SASAIMGNPK
  61 VKAHGKKVLT SLGDAIKHLD DLKGTFAQLS
        ELHCDKLHVD PENFKLLGNV LVTVLAIHFG
121 KEFTPEVQAS WQKMVTGVAS ALSSRYH,
```
or a sequence having at least 90% identity to the sequence of SEQ ID NO: 5 or
wherein the subunit γ is encoded by the nucleic acid sequence of
```
(SEQ ID NO: 6)
    1 acactcgctt ctggaacgtc tgaggttatc
        aataagctcc tagtccagac gccatgggtc
  61 atttcacaga ggaggacaag gctactatca
        caagcctgtg gggcaaggtg aatgtggaag
121 atgctggagg agaaaccctg ggaaggctcc
        tggttgtcta cccatggacc cagaggttct
181 ttgacagctt tggcaacctg tcctctgcct
        ctgccatcat gggcaacccc aaagtcaagg
241 cacatggcaa gaaggtgctg acttccttgg
        gagatgccat aaagcacctg gatgatctca
301 agggcacctt tgcccagctg agtgaactgc
        actgtgacaa gctgcatgtg gatcctgaga
361 acttcaagct cctgggaaat gtgctggtga
        ccgttttggc aatccatttc ggcaaagaat
421 tcacccctga ggtgcaggct tcctggcaga
        agatggtgac tggagtggcc agtgccctgt
481 cctccagata ccactgagct cactgcccat
        gatgcagagc tttcaaggat aggctttatt
541 ctgcaagcaa tcaaataata aatctattct
        gctaagagat cacaca,
```
or a sequence having at least 90% identity to the sequence of SEQ ID NO: 6; or
(d) a subunit gamma (γ), wherein the subunit γ comprises the amino acid sequence of
```
(SEQ ID NO: 7)
    1 MGHFTEEDKA TITSLWGKVN VEDAGGETLG
        RLLVVYPWTQ RFFDSFGNLS SASAIMGNPK
  61 VKAHGKKVLT SLGDATKHLD DLKGTFAQLS
        ELHCDKLHVD PENFKLLGNV LVTVLAIHFG
121 KEFTPEVQAS WQKMVTAVAS ALSSRYH,
```
or a sequence having at least 90% identity to the sequence of SEQ ID NO: 7 or
wherein the subunit γ is encoded by the nucleic acid sequence of
```
(SEQ ID NO: 8)
    1 acactcgctt ctggaacgtc tgaggttatc
        aataagctcc tagtccagac gccatgggtc
  61 atttcacaga ggaggacaag gctactatca
        caagcctgtg gggcaaggtg aatgtggaag
121 atgctggagg agaaaccctg ggaaggctcc
        tggttgtcta cccatggacc cagaggttct
181 ttgacagctt tggcaacctg tcctctgcct
        ctgccatcat gggcaacccc aaagtcaagg
241 cacatggcaa gaaggtgctg acttccttgg
        gagatgccac aaagcacctg gatgatctca
301 agggcacctt tgcccagctg agtgaactgc
        actgtgacaa gctgcatgtg gatcctgaga
361 acttcaagct cctgggaaat gtgctggtga
        ccgttttggc aatccatttc ggcaaagaat
421 tcacccctga ggtgcaggct tcctggcaga
        agatggtgac tgcagtggcc agtgccctgt
481 cctccagata ccactgagct cactgcccat
        gattcagagc tttcaaggat aggctttatt
541 ctgcaagcaa tacaaataat aaatctattc
        tgctgagaga tcac,
```
or a sequence having at least 90% identity to the sequence of SEQ ID NO: 8.

26. The composition of any one of claims 1-25, wherein the composition is stable at an ambient temperature.

27. The composition of any one of claims 1-26, wherein the composition is stable at a refrigerated temperature.

28. The composition of any one of claims 1-27, wherein the composition is stable above a temperature of at least 4° C.

29. The composition of any one of claims 1-28, wherein the composition is stable below a temperature of 30° C.

30. The composition of any one of claims 13-29, wherein the endotoxins comprise a cellular lipid, a cellular lipid layer, or a lipopolysaccharide.

31. The composition of claim 30, wherein the cellular lipid, cellular lipid layer, or lipopolysaccharide is from a human cell.

32. The composition of claim 30, wherein the cellular lipid, cellular lipid layer, or lipopolysaccharide is from a non-human vertebrate cell.

33. The composition of claim 30, wherein the cellular lipid, cellular lipid layer, or lipopolysaccharide is from a microbe.

34. The composition of claim 33, wherein the cellular lipid, cellular lipid layer, or lipopolysaccharide is from a bacterium.

35. The composition of any one of claims 1-34, wherein the stabilized hemoglobin is non-naturally occurring.

36. The composition of any one of claims 1-35, wherein the stabilized hemoglobin is polymerized.

37. The composition of claim 36, wherein the stabilized hemoglobin is cross-linked with an aldehyde to form a hemoglobin glutamer.

38. The composition of claim 37, wherein the aldehyde is glutaraldehyde.

39. The composition of any one of claims 1-38, wherein the stabilized hemoglobin has an average molecular weight of 70-200 kilodaltons (kDa).

40. The composition of any one of claims 1-39, wherein the stabilized hemoglobin has less than 15% molecular weight distribution over 500 kDa.

41. The composition of any one of claims 1-40, wherein the stabilized hemoglobin has been substantially deoxygenized prior to stabilization with a stabilizing agent.

42. The composition of claim 41, wherein the stabilization comprises polymerization.

43. The composition of claim 41 or 42, wherein the stabilization comprises reduction of the stabilizing agent.

44. The composition of any one of claims 1-43, wherein the stabilized hemoglobin is concentrated by filtration and/or diafiltration with an electrolyte solution.

45. The composition of claim 44, wherein the electrolyte solution is a physiologic electrolyte solution.

46. The composition of claim 44 or 45, wherein the filtration is ultrafiltration.

47. The composition of claim 45, wherein the electrolyte solution minimizes formation of methemoglobin (MetHb).

48. The composition of any one of claims 44-47, wherein the electrolyte solution comprises N-acetyl-L-cysteine.

49. The composition of any one of claims 1-48, wherein the composition comprises:
(a) less than 10% MetHb, optionally less than 6% MetHb; and/or
(b) less than 10% hemoglobin dimers, optionally less than 6% hemoglobin dimers.

50. The composition of claim 49, wherein the level of MetHb is measured by cooximetry.

51. The composition of claim 49, wherein the level of hemoglobin dimers is measured by a size separation technique.

52. The composition of any one of claims 1-51, wherein the composition comprises at least 20% stabilized active tetrameric hemoglobin, optionally 25% to 35% stabilized active tetrameric hemoglobin.

53. The composition of any one of claims 1-52, wherein the composition comprises at least 60% greater-than-tetrameric molecular weight hemoglobin oligomers, optionally at least 70% greater-than-tetrameric molecular weight hemoglobin oligomers.

54. The composition of any one of claims 1-53, wherein the stabilized hemoglobin has a longer half-life than non-stabilized or oxygenated hemoglobin and minimizes breakdown of tetrameric hemoglobin into dimers that cause renal toxicity.

55. The composition of any one of claims 1-54, wherein the stabilized hemoglobin comprises at least one subunit that is synthesized in vitro.

56. The composition of claim 55, wherein the at least one subunit comprises a gamma (Y) subunit.

57. The composition of any one of claims 1-56, wherein the stabilized hemoglobin is not isolated from a human fetus.

58. A pharmaceutical formulation comprising the composition of any one of claims 1-57, wherein the pharmaceutical formulation further comprises a pharmaceutically-acceptable excipient, a pharmaceutically-acceptable solvent or a pharmaceutically-acceptable carrier.

59. The pharmaceutical formulation of claim 58, wherein the composition is formulated for intravenous injection.

60. The pharmaceutical formulation of claim 58, wherein the composition is formulated for intraosseous injection.

61. An injection device comprising the composition of any one of claims 1-57.

62. An injection device comprising the pharmaceutical formulation of any one of claims 58-60.

63. The injection device of claim 61 or 62, wherein the device comprises one or more of a needle, an injection pen, an intravenous (IV) line, a central IV line, a syringe, a catheter, and a blood exchanging and/or filtering device.

64. The injection device of any one of claims 61-63, wherein the device is intended for administration by an individual who is not a medical professional.

65. The injection device of claim 64, wherein the device comprises a preloaded self-injection device.

66. The injection device of any one of claims 62-65, wherein the device comprises one or more therapeutically effective doses of the pharmaceutical formulation.

67. The injection device of any one of claims 62-65, wherein the device comprises one or more unit doses of the pharmaceutical formulation.

68. The injection device of claim 67, wherein the one or more unit doses comprise of volume of between 10 mL and 30 mL, inclusive of the endpoints.

69. The injection device of claim 68, wherein the one or more unit doses comprise of volume of between 18 mL and 25 mL, inclusive of the endpoints.

70. The injection device of any one of claims 61-69, wherein the injection device comprises a metering device.

71. The injection device of any one of claims 61-70, wherein the injection device is operably linked to a metering device.

72. The injection device of any one of claims 61-71, wherein the injection device may be connected to a metering device.

73. The injection device of any one of claims 61-72, wherein the injection device comprises a titrated dose.

74. The injection device of any one of claims 61-73, wherein the injection device comprises one or more compartments, each capable of maintaining a preloaded volume of the composition of any one of claims 1-57, and each capable of delivering to a subject a distinct volume of the composition, wherein the volume of the composition in each compartment may be delivered simultaneously or sequentially.

75. The injection device of any one of claims 61-74, wherein the injection device comprises one or more compartments, each capable of comprising a distinct amount of the formulation buffer to selectively dilute the composition to a predetermined final concentration for each compartment.

76. The use of the composition of any one of claims 1-57 for the treatment of a subject in need thereof.

77. The use of the pharmaceutical formulation of any one of claims 58-60 for the treatment of a subject in need thereof.

78. The use of the injection device of any one of claims 61-75 for the treatment of a subject in need thereof.

79. The use of any one of claims 76-78, wherein the subject is hypoxic and/or anemic.

80. The use of any one of claims 76-79, wherein the subject has experienced blood loss from an injury, blood loss from a medical intervention, hemolysis or reduced hematopoiesis.

81. The use of any one of claims 76-80, wherein the subject is a human.

82. The use of any one of claims 76-80, wherein the subject is a non-human animal.

83. The use of claim 82, wherein the non-human animal is a non-human vertebrate, a non-human primate, a cetacean, a mammal, a reptile, a bird, an amphibian, or a fish.

84. The use of claim 82 or 83, wherein the non-human animal is a bovine.

85. The use of claim 82 or 83, wherein the non-human animal is a mustelid, a captive mustelid, a rodent, a captive rodent, a raptor, or a captive bird.

86. The use of claim 85, wherein the captive bird is of the order Psittaciformes, Passeriformes or Columbiformes.

87. A method of treating a condition comprising administering to a subject in need thereof the composition of any one of claims 1-57.

88. The method of claim 87, wherein the administering comprises providing a therapeutically effective amount of the composition to the subject in one or more doses.

89. A method of treating a condition comprising administering to a subject in need thereof the pharmaceutical formulation of any one of claims 58-60.

90. The method of claim 89, wherein the administering comprises providing a therapeutically effective amount of the pharmaceutical formulation to the subject in one or more doses.

91. A method of treating a condition comprising providing to a subject in need thereof the injection device of any one of claims 61-75, wherein the device injects the composition into the subject, thereby treating the subject.

92. The method of claim 91, wherein the injection device comprises a therapeutically effective amount of the composition.

93. The method of claim 91 or 92, wherein the injection device comprises one or more doses of the composition.

94. The method of any one of claims 91-93, wherein the injection device provides an escalating or de-escalating dosage regime by injecting from each of the one or more compartments, sequentially,
(a) an increasing or a decreasing volume of the composition of any one of claims 1-57, respectively, or
(b) an increasing or a decreasing concentration of the composition of any one of claims 1-57, respectively.

95. The method of any one of claims 91-94, wherein the injection device provides an escalating or de-escalating dosage regime by injecting from each of the one or more compartments, sequentially,
(a) an increasing or a decreasing volume of the pharmaceutical formulation of any one of claims 51-53, respectively, or
(b) an increasing or a decreasing concentration of the pharmaceutical formulation of any one of claims 51-53, respectively.

96. The method of any one of claims 87-95, wherein the condition is hypoxia and/or anemia.

97. The method of any one of claims 87-96, wherein the subject is hypoxic and/or anemic.

98. The method of claim 97, wherein the subject has experienced blood loss from an injury, blood loss from a medical intervention, hemolysis or reduced hematopoiesis.

99. The method of any one of claims 87-98, wherein the subject is a human.

100. The method of any one of claims 87-98, wherein the subject is a non-human animal.

101. The method of claim 100, wherein the non-human animal is a non-human vertebrate, a non-human primate, a cetacean, a mammal, a reptile, a bird, an amphibian, or a fish.

102. The method of claim 100 or 101, wherein the non-human animal is a bovine.

103. The method of claim 100 or 101, wherein the non-human animal is a mustelid, a captive mustelid, a rodent, a captive rodent, a raptor, or a captive bird.

104. The method of claim 103, wherein the captive bird is of the order Psittaciformes, Passeriformes or Columbiformes.

105. The method of any one of claims 87-104, wherein the composition, pharmaceutical formulation, or injection is administered to the subject on a repeated dosing schedule.

106. The method according to claim 105, wherein the repeated doses are administered to achieve and/or maintain a plasma concentration of 0.3-0.4 g/dL of stabilized hemoglobin.
